(19) United States
(12) Patent Application Publication
(10) Pub. No.: US 2018/0306780 A1
(43) Pub. Date: Oct. 25, 2018

(54) ASTROCYTE DIFFERENTIATION PROTOCOL

(71) Applicant: The Regents of the University of California, Oakland, CA (US)

(72) Inventors: Alysson Renato Muotri, La Jolla, CA (US); Beatriz Camille Grigalis de Freitas, San Diego, CA (US)

(21) Appl. No.: 15/767,924

(22) PCT Filed: Oct. 27, 2016
(86) PCT No.: PCT/US16/59201

§ 371 Date: Apr. 12, 2018

Related U.S. Application Data
(60) Provisional application No. 62/248,224, filed on Oct. 29, 2015.

(51) Int. Cl. G01N 33/50; C12N 5/074; G01N 33/68; C12N 5/079

(52) U.S. Cl. CPC G01N 33/5058; C12N 5/0696; G01N 33/6896; C12N 5/0622; A61P 25/18; G01N 2800/2814; C12N 2501/155; C12N 2506/45; C12N 2506/1307; C12N 2501/113

(57) ABSTRACT

The disclosure provides for methods of compositions for making astrocytes from induced pluripotent stem cells as well as method of treating and diagnosing neurological disorders such as Rett syndrome.

### CROSS REFERENCE TO RELATED APPLICATIONS

This application claims priority under 35 U.S.C. § 119 from Provisional Application Ser. No. 62/248,224, filed Oct. 29, 2015, the disclosure of which is incorporated herein by reference.

### TECHNICAL FIELD

The disclosure is directed generally to stem cells, method of making and using such cells. More particularly, the disclosure is directed to methods of manipulating the differentiation of stem cells.

### BACKGROUND

The study of autism spectrum disorder (ASD) risk variants is critical for the understanding of autism pathophysiology. Induced pluripotent stem cells (iPSCs) provide a valuable strategy to study the effects of these variants in living patient cells. While models have been developed for monogenic forms of ASD, no models of idiopathic ASD using iPSCs have heretofore been reported.

Rett syndrome (RTT) is a devastating disease that affects 1 in every 10,000 children born in the United States, primarily females. RTT patients undergo apparently normal development until 6-18 months of age, followed by impaired motor function, stagnation and then regression of developmental skills, hypotonia, seizures and a spectrum of autistic behaviors. Rett syndrome is a rare disease that shares certain pathways with major developmental disorders such as autism and schizophrenia, increasing the potential impact. There is no cure for Rett syndrome and the animal model does not entirely recapitulate the human disease. Thus, having the possibility to screen drugs directly in human neurons is a major milestone.

### SUMMARY

The disclosure provides a cell culture method for generating astrocytes comprising culturing neuronal precursor cells (NPCs) in an NGF media to confluence, washing and incubating the NPCs with dPBS, then scraping the NPCs to form neurospheres in NGF media; dissociating, plating and culturing the neurospheres; adding ROCK inhibitor in FGF-free NGF media and culturing for 1 to 3 days; culturing the neurospheres in FGF-free NGF media without ROCK inhibitor for about 1 week; subculturing the neurospheres in astrocyte media for about 2 weeks while shaking; and isolating astrocytes that project outside of the spheres; and subculturing the isolated astrocytes. In one embodiment, the NPCs are obtained from induced pluripotent stem cells. In a further embodiment, the induced pluripotent stem cells are obtained from fibroblasts. In still a further embodiment, the fibroblasts are obtained from a subject having a neuronal disease, disorder or syndrome (e.g., Rett syndrome). In another embodiment, the NGF media comprises DMEM/F12 media supplemented with 0.5×N2, 0.5× Gem21 supplement, 20 ng/mL of FGF and 1% penicillin/streptomycin.

The disclosure also provides a population of astrocytes obtained by the methods described above.

The disclosure also provides a method comprising contacting a human somatic cell obtained from a subject having with at least one retroviral vector, wherein each of the at least one retroviral vectors comprises one or more polynucleotides encoding at least four de-differentiation factors selected from the group consisting of a (i) KLF4, (ii) OCT4, (iii) SOX2, (iv) c-MYC or n-MYC, and (v) NANOG; culturing the somatic cell to express the de-differentiation factor; selecting cells displaying a pluripotent phenotype; subculturing the selected cells to obtain an enriched population of de-differentiated induced pluripotent stem cells (iPSCs); culturing the iPSCs to form embryoid bodies (EBs); culturing the EBs in N2 media with dual SMAD inhibitors (Dorsomorphin and SB) with constant shaking and selecting rosettes for subculture; subculturing the rosettes in NGF media until NPCs are formed; changing the NGF media as needed until neurospheres are formed providing a neurosphere culture; adding Rock inhibitor to the neurospheres for 48 hours concomitant with the removal of FGF from the media; removing Rock inhibitor and culturing the neurospheres in neuronal media without FGF for about one week; washing the neurospheres and culturing in astrocyte growth media for about two weeks with shaking; plating neurospheres with astrocyte projections; isolating astrocytes.

The disclosure also provides a method of treating Rett syndrome comprising inhibiting IL-6 production in the neuronal cells of a subject having Rett syndrome.

The disclosure also provides a method of identifying a neurological disorder or Rett syndrome in a subject comprising measuring the level of IL-6 production from astrocyte obtained by the method of claim 7 wherein an elevated amount of IL-6 compared to a control is indicative of Rett syndrome or neurological disorder.

### DESCRIPTION OF DRAWINGS

FIG. 1A-I shows generation of human iPSC-derived neurons. (A) Schematic representation of the MECP2 gene and mutations used. The untranslated regions (UTR), methyl-CpG-binding domain (MBD), transcriptional repression domain (TRD) and WW domain are represented, illustrating main features of the MECP2 gene. The schematic tree illustrates the samples generated by iPSCs or isogenic cells (CRISPR). Mutated rescue of Q83X patient is labeled rRTT (rQ83X) in the results and mutations in hESCs are labeled RTT/C9. hESCs submitted to the CRISPR/Cas9 procedure but without effective mutation were labeled Ctrl/C9. (B) Volume and area reduction in the soma of RTT-derived neurons compared to controls. Data are represented as mean±SEM. (C) Misregulation of gene expression in several pathways in RTT neurons compared to control. (D) Representative image of a co-localized punch indicated by arrow. (E) Reduction in co-localized SYN1 and Homerl puncta. Data are represented as mean±SEM. (F) Representative image of a single electrode from the MEA plate. (G) Spike rate (spikes per second) comparing control neurons with RTT and rRTT. Data are represented as mean±SEM. (H) Number of spikes per seconds in the MEA readings from each patient line. (I) Representative raster plot image with the number of bursts per 5 minutes in each cell line tested.

FIG. 2A-F shows RTT astrocyte phenotypes in vitro and impact in human neurons. (A) Gene expression of relevant astrocyte genes and pathways misregulated in RTT astrocytes. Data are represented as mean±SEM and t-test result values are: (**) p≤0.005; (***) p≤0.001; (****) p<0.0001. (B) Calcium wave propagation is defective when RTT-derived astrocytes are mechanically stimulated. Data are represented as mean±SEM. (C) Control human astrocytes can propagate a calcium wave when mechanically stimulated. A single cell being mechanically stimulated; the elapsed time is 12 seconds. (D) Immunofluorescence of the co-culture model showing the layer of astrocytes stained with GFAP and neuronal MAP2 identifying a neuron between astrocytes. Branches and spines arising from the dendrites are visible (arrows). (E) Quantification of the total dendritic length (TDL), dendritic segment number and the total number of dendritic spines in each condition. Data are represented as mean±SEM. (F) Representative images of MAP2-stained neurons co-cultured on each astrocyte type.

FIG. 3A-H shows IL-6 release from astrocyte RTT cultures in the absence of exogenous stimulus. (A) Significant IL-6 release from RTT-derived astrocytes rescued when MECP2 is re-expressed in the cell. Data are represented as mean±SEM. (B) RTT/C9 isogenic cells release significant levels of IL-6 to the media compared to controls. Human embryonic cells H1 and H9 are grouped as hESCs. Data are represented as mean±SEM. (C) Elevated ROS and (D) GSH levels in RTT-derived astrocytes compared to control cells. Data are represented as mean±SEM. (E) RTT-derived astrocytes release significant more TNFα, IL-4, IL-13 and IL-10 compared to controls. Data are represented as mean±SEM. (F) IL-6 receptor is expressed in iPSC-derived astrocytes and neurons but not in NPCs. Data are represented as mean±SEM. (G) A RTT post-mortem rare male sample has increased IL-6 and IL6ST (gp130) expression. Data are represented as mean±SEM. (H) Levels of IL-6 protein in RTT post-mortem brain sample are significantly elevated compared to age matched control. Data are represented as mean±SEM.

FIG. 4A-G shows exogenous IL-6 levels can impact human neurons. (A) Synapsin 1 puncta is reduced in control neurons treated with IL-6. Data are represented as mean±SEM. (B) Control neurons are smaller with a total dendritic length (TDL) significantly reduced when exposed to IL-6 in the media. Data are represented as mean±SEM. (C) and (D) Shows that the addition of IL-6 in control neurons using MEA reduces their spike rate and their total number of spikes. Data are represented as mean±SEM. (E) IGF-1 levels are reduced both in iPSC-derived neurons or RTT post-mortem brain sample. Data are represented as mean±SEM. (F) IL-6 gene expression is reduced in control neurons only when IGF-1 is added to the media. Data are represented as mean±SEM. (G) The IGF-1 carrier IGFBP3 expression is induced when neurons are treated with IL-6. Data are represented as mean±SEM.

FIG. 5A-U shows the generation of iPSC and isogenic lines related to FIG. 1. (A) Global gene expression analysis of iPSCs, fibroblast, primary human astrocyte from Lonza (NHA), hES-derived astrocyte (H9-AS) and iPS-derived control astrocyte (iPSAS). Highlighted are the pluripotent genes highly expressed in H9 and iPSCs but not in fibroblasts. (B) Representative immunofluorescence staining of pluripotent markers Nanog, Sox2, Oct4 and Lin28 in one clone of our patient cell line cohort. Scale bar, 20 μm. (C) Representative G-banding karyotyping from WT83 and Q83X cell lines. (D) PCR comparing the expression between iPSCs and embryoid bodies (EB) obtained from their respective iPSCs of three germ layers (ectoderm, mesoderm and endoderm) markers (PAX6, MSX1 and AFP). (E) Schematic overview of the CRISPR/Cas-9 induced MECP2 mutation. DNA sequence chromatogram shows the nucleotide deletion in the MeCP2 gene leading to a frameshift mutation (K82fs) and a predicted premature stop-codon in the end of exon 3 asterisk). The WT83 and Q83X iPSC lines are shown for reference. Line represents the guide RNA target locus. (SEQ ID Nos:1-3) (F) Gel images showing Surveyor nuclease assay of genomic DNA extracted from FACs sorted H9 hESC. Expected PCR product was 278 bp and 220 bp. (G) Immunofluorescence of isogenic pairs of MeCP2 mutated and control cell lines colony showing the expression of the pluripotent marker Nanog. Scale bar, 100 μm. (H) Eosin and Hematoxylin stains of teratomas showing the presence of all three germ layers. Scale bar, 200 μm. (I) Western blot of the isogenic pluripotent stem cells showing the absence of MeCP2 in the mutated lines. (J) Karyotypes of MeCP2 mutated cell lines displaying no chromosomal abnormality. (K-L) Expression of pluripotent markers and MeCP2 by qPCR. GAPDH was used as housekeeping gene. (M) Representative immunofluorescence of MeCP2 staining in control (WT83), RTT (Q83X) and rRTT. Scale bar, 20 μm. (N) Flow characterization of expression of SOX1, Nestin, PAX6 and SOX2 in our NPC population. (O) Representative immunofluorescence of NPCs expressing Nestin, Sox2 and Musashil. Scale bar, 20 μm. (P) Cortical layer marker CTIP2 and SATB2 in a representative image of a control (WT83) neuronal culture. (Q) Gene expression analysis of genes regulating neuronal differentiation. Two control NPCs and two clones of control cells were used for this representative characterization. (R) Flow analysis of the expression of GFAP and CD24 in our neuronal differentiation protocol between cell lines. (S) Protein quantification of PSD-95, SYN1, MAP2 and MeCP2 in the neuronal cultures when kept for 4 weeks differentiating. GAPDH is our loading control. (T) SYN1 and PSD-95 in cell western levels measured after 4 weeks of differentiation. (U) Rescue of SYN1 levels measured by in cell western after addition of IGF-1 to the media for two weeks after two weeks of differentiation.

FIG. 6A-K shows the generation of iPS-derived astrocytes related to FIG. 2. (A) An exemplary chematic timeline of the astrocyte differentiation method. (B) Brightfield images of the main steps during astrocyte differentiation. Scale bars, 1000 μm. (C-D) Representative immunofluorescence of astrocytes stained with GFAP, s100β and DAPI (nuclear marker) after plating (P0, C) and at passage 3 (D). Scale bars, 100 μm. (E) RNAseq cluster expression analysis comparing known astrocyte-related genes between NHA, H9- and iPS-derived astrocytes. The main and most expressed genes are represented by the column graphs separated by amount of expression of repeats: 10 k, 1 k, 550 and 50 indicated by the red lines followed by a transcriptional factor graph comparing the samples identities. (F) Fluorescence-based flow cytometry analysis showing that human iPSC-derived astrocytes differ from neural progenitor cells (NPCs) and neurons based on their high expression of CD44. (G) Schematic representation of the co-culture strategy. Neural progenitor cells (NPCs) can be differentiated toward a neuronal fate after contaminant cells are removed by sorting. Following sorting, the enriched neuronal population is placed directly on astrocytes for 2 weeks prior to fixation. (H) Schematic representation of a neuron and its parts: soma, spines, dendrite, dendrite tree and dendritic segment orders (indicated by numbers). (I) An example of neuronal tracings comparing controls and RTT neurons. Scale bar, 10 μm. (J) The mean segment length per segment order in each condition tested. (K) The spine percentage in each segment order from each condition. P values are: (*) p<0.04. (**) p<0.005.

FIG. 7A-D shows cytokine expression and modulation in iPSCs related to FIG. 3. (A) CHIP assay showing the occupancy of MeCP2 in GDNF, IL-6 and BDNF promoter regions. (B) Absolute read count and normalized expression values (in RPKM) of cytokine receptors in RNA-seq of sorted iPSC control neurons. (C) IL-6 media levels from RTT and isogenic cell lines are comparable. (D) Caspase 3/7 assay in RTT and control astrocytes.

FIG. 8 shows a schematic drawing of the IL-6 regulation and effects in neuronal homeostasis. MeCP2 can regulate the transcription of IL-6. Besides impairing the IGF-1 transcription, IL-6 also increases the IGFBP3, limiting the availability of IGF-1 for neurons, causing a detrimental effect on PSD-95 and SYN1 levels. Other cytokine signaling with IL-4, IL-10 and IL-13 could have a specific neuro-inflammatory effect either signaling other immune cells or affecting neuronal viability.

### DETAILED DESCRIPTION

As used herein and in the appended claims, the singular forms “a,” “and,” and “the” include plural referents unless the context clearly dictates otherwise. Thus, for example, reference to “a cell” includes a plurality of such cells and reference to “the mutation” includes reference to one or more mutations known to those skilled in the art, and so forth.

Unless defined otherwise, all technical and scientific terms used herein have the same meaning as commonly understood to one of ordinary skill in the art.

Also, the use of “or” means “and/or” unless indicated otherwise, such as by the use of the term “either.” Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated by reference in full for the purpose of describing and disclosing methodologies that might be used in connection with the description herein. Moreover, with respect to any term that is presented in the publications that is similar to, or identical with, a term that has been expressly defined in this disclosure, the definition of the term as expressly provided in this disclosure will control in all respects.

Rett Syndrome (RTT) is a devastating disease that affects 1 in every 10,000 children, primarily females, in the United States. Rett Syndrome (RTT) is a severe neurological disorder caused by mutations in the X-linked methyl-CpG-binding protein 2 (MeCP2) gene. Although previous studies suggest that astrocytes play an important role in neuronal homeostasis, how the absence of MeCP2 in human astrocytes affect neuronal development is unknown. Currently, there are no known preventative approaches or therapeutic treatments available for RTT. RTT patients undergo seemingly normal development until 6-18 months of age, where the onset of motor impairments, stagnation, regression of developmental skills, hypotonia, seizures and a spectrum of autistic behaviors begin to occur. The development of autistic features, such as social withdrawal and loss of eye contact, characterize the behavioral and motor regression phase of RTT. Alterations in the MeCP2 gene clearly impair the physiology of human neurons, and experiments using mouse models, and iPSC-derived mouse astrocytes indicate that the loss of MeCP2 protein function in astrocytes negatively affects neurons in a non-cell-autonomous fashion.

Clinical similarities in behavior phenotypes between the regressive phase in RTT and other ASD patients suggest a common underlying biological process. One critical link between ASD and RTT is an imbalance in cytokine signaling. IL-6 has been associated with ASD in mouse models; and a single injection of IL-6 in a pregnant mouse is sufficient to produce offspring with autistic features. Furthermore, IL-6 has been linked to alterations in ASD patients' brains, affecting neural cell adhesion, migration, and synapse maturation. Previous experiments investigating the cell types responsible for the detrimental effects of IL-6 in the brain, showed that selective IL-6 overexpression in mouse astrocytes revealed robust ASD behavioral features. Immunological impairments have been reported in RTT, specially focusing on interleukin abnormalities, which include elevated levels of IL-6. Importantly, cytokine signaling can differ between mice and humans, and the ability to rapidly profile different mutations in relevant cell types highlights the value of the human iPSC system to complement current disease modeling efforts. Further investigation of the mechanisms involved in the release of signaling cytokines and their regulation by MeCP2 in glial cells is necessary for a better understanding of RTT pathology. The efficient astrocyte-differentiation method described here, together with the insights on RTT astrocytes, can be applied to other neurological disorders with a non-cell autonomous neuro-inflammatory component.

The mechanisms through which astrocytes affect neurons in human RTT models are still unknown, particularly due to the complications in obtaining human astrocytes to study. Protocols for human astrocyte differentiation from iPSCs are labor intensive and require the extended use of a combination of exogenous cytokines (such as Ciliary Neurotrophic Factor or murine Leukemia inhibitory factor) for several weeks, potentially masking important differences in astrocyte development between disease and control cells. To address these shortcomings, the disclosure provides a fast protocol to efficiently generate human astrocytes from iPSCs by taking advantage of the presence of neurons in three dimensional clusters which can secrete factors that promote astrocyte differentiation, as naturally occurs during neurodevelopment. Using this protocol, an IL-6-mediated mechanism was identified in IPSC-derived human astrocytes by which RTT-derived astrocytes negatively impact cortical neurons, offering unexplored therapeutic opportunities for RTT and potentially to other autism spectrum disorders (ASD).

To determine how human astrocytes contribute to RTT, a highly efficient method for generating astrocytes from induced pluripotent stem cells (iPSCs) is provided. Using a well-characterized rare male RTT patient-derived and genome-edited isogenic lines, an enriched population of cortical neurons and astrocytes was generated. RTT-derived astrocytes showed significant gene expression and physiological differences compared to control cells. Co-culture experiments revealed that RTT-derived astrocytes impaired neuronal dendritic arborization and spinogenesis in control cells. In contrast, control-derived astrocytes rescued morphological RTT neuronal phenotypes. In RTT-derived astrocytes, elevated secretion of interleukin 6 (IL-6) was identified, without exogenous stimulation, which when applied to control neurons can significantly reduce the neuronal length and synaptogenesis thus reducing the rate of spontaneous electrical spikes. These findings demonstrate the reconstitution of a complex human neuronal-astrocytic system in which the mechanistic contribution of specific cell types to RTT can be distinguished.

The disclosure thus provides the efficient differentiation of induced pluripotent stem cells, neuronal stem cells or neuronal precursor cells to an astrocyte cell type and demonstrates their use to model and screen disease states.

For example, human fibroblast (or other somatic cells) can be isolated from a subject and de-differentiated to induced pluripotent stem cells (iPSCs). The disclosure uses a plurality of de-differentiation factors for de-differentiating lineage committed cells to a more pluripotent or omnipotent cell type. As used herein a de-differentiation factor comprises a polynucleotide, polypeptide or small molecule. Exemplary de-differentiation factors comprising a polynucleotide are selected from the group consisting of a polynucleotide encoding a NANOG polypeptide, a c-MYC or n-MYC polypeptide, a KLF4 polypeptide, a SOX2 polypeptide or OCT4 polypeptide. Exemplary polypeptides comprise NANOG, c-MYC or n-MYC, KLF4, SOX2 or OCT4 polypeptides or polypeptides that increase the expression of any of the foregoing. Useful small molecule de-differentiation factors include molecules that stimulate the transcription or activity of an endogenous Nanog, c-Myc or n-Myc, Klf4, Sox4 or Oct4 polynucleotide or polypeptide, respectively.

A method to de-differentiate cells by expression of KLF4, OCT4, SOX2, c-MYC or n-MYC, NANOG or any combination thereof is presented. The nucleic acid and amino acid sequences of mouse and human KLF4, OCT4, SOX2, c-MYC or n-MYC, NANOG or any combination thereof are known in the art. The disclosure demonstrates that transfection with KLF4, OCT4, SOX2, c-MYC or n-MYC, and NANOG results in a de-differentiation of committed fibroblasts (e.g., dermal fibroblasts) to a pool of proliferating stem cells that are capable of redifferentiating into several cell types (including lineage committed neuronal cells).

In addition to the expression of a nucleic acid encoding an KLF4, OCT4, SOX2, c-MYC or n-MYC, and/or NANOG polypeptide, the disclosure contemplates that any agent which increase the expression and/or activity of an endogenous KLF4, OCT4, SOX2, c-MYC or n-MYC, NANOG or any combination thereof can be used in the methods of the disclosure to promote de-differentiation.

The term “precursor cell,” “progenitor cell,” and “stem cell” are used interchangeably in the art and herein and refer either to a pluripotent, or lineage-uncommitted, progenitor cell, which is potentially capable of an unlimited number of mitotic divisions to either renew its line or to produce progeny cells which will differentiate into fibroblasts or a lineage-committed progenitor cell and its progeny, which is capable of self-renewal and is capable of differentiating into a parenchymal cell type. Unlike pluripotent stem cells, lineage-committed progenitor cells are generally considered to be incapable of giving rise to numerous cell types that phenotypically differ from each other. Instead, they give rise to one or possibly two lineage-committed cell types.

The term “de-differentiation” is familiar to the person skilled in the relevant art. In general de-differentiation signifies the regression of lineage committed cell to the status of a stem cell, for example, by “inducing” a de-differentiated phenotype. For example, as described further herein KLF4, OCT4, SOX2, c-MYC or n-MYC, and Nanog can induce de-differentiation and induction of mitosis in lineage committed mitotically inhibited cells.

Nanog is a gene expressed in embryonic stem cells (ESCs) and plays a role in maintaining pluripotency. NANOG is thought to function with SOX2. Human NANOG protein (see, e.g., Accession number NP_079141, incorporated herein by reference) is a 305 amino acid protein with a homeodomain motif that is localized to the nuclear component of cells. Similar to murine NANOG, N-terminal region of human NANOG is rich in Ser, Thr and Pro residues and the C-terminus comprises Trp repeats. The homeodomain in human NANOG ranges from about residue 95 to about residue 155. Homologs of human NANOG are known.

Oct-4 (Octamer-4) is a homeodomain transcription factor of the POU family and regulates the expression of numerous genes (see, e.g., J. Biol. Chem., Vol. 282, Issue 29, 21551-21560, Jul. 20, 2007, incorporated herein by reference). Homologs of human Oct-4 are known as set forth in the following accession numbers NP_038661.1 and NM_013633.1 (Mus musculus), NP_001009178 and NM_001009178 (Rattus norvegicus), and NP_571187 and NM_131112 (Danio rerio), which are incorporated herein by reference.

SRY (sex determining region Y)-box 2, also known as SOX2, is a transcription factor that plays a role in self-renewal of undifferentiated embryonic stem cells and transactivation of Fgf4 as well as modulating DNA bending (see, e.g., Scaffidi et al. J. Biol. Chem., Vol. 276, Issue 50, 47296-47302, Dec. 14, 2001, incorporated herein by reference). Homologs of human SOX2 are known.

Kruppel-like factor 4, also known as KLF4 plays a role in stem cell maintenance and growth. Homologs of human KLF4 are known and include NP_034767, NM_010637 (Mus musculus), which are incorporated herein by reference.

The MYC family of cellular genes is comprised of c-myc, N-myc, and L-myc, three genes that function in regulation of cellular proliferation, differentiation, and apoptosis (Henriksson and Luscher 1996; Facchini and Penn 1998). Although myc family genes have common structural and biological activity. N-Myc is a member of the MYC family and encodes a protein with a basic helix-loop-helix (bHLH) domain. The genomic structures of C-myc and N-myc are similarly organized and are comprised of three exons. Most of the first exon and the 3′ portion of the third exon contain untranslated regions that carry transcriptional or post-transcriptional regulatory sequences. N-myc protein is found in the nucleus and dimerizes with another bHLH protein in order to bind DNA. Homologs and variants of the Myc family of proteins are known in the art.

cDNA coding for the human oct4 (pour5f1), sox2, klf4, c-myc (or n-myc) and nanog, variants and homologs thereof can be cloned and expressed using techniques known in the art. Using the sequences set forth in the accession numbers above and available to one of skill in the art, one or more de-differentiation factors can be cloned into a suitable vector for expression in a cell type of interest.

Cells can be engineered using any of a variety of vectors including, but not limited to, integrating viral vectors, e.g., retrovirus vector or adeno-associated viral vectors; or non-integrating replicating vectors, e.g., papilloma virus vectors, SV40 vectors, adenoviral vectors; or replication-defective viral vectors. Where transient expression is desired, non-integrating vectors and replication defective vectors may be used, since either inducible or constitutive promoters can be used in these systems to control expression of the gene of interest. Where the vector is a non-integrating vectors, such vectors can be lost from cells by dilution after reprogramming, as desired. An example of a non-integrating vector includes Epstein-Barr virus (EBV) vector. Alternatively, integrating vectors can be used to obtain transient expression, provided the gene of interest is controlled by an inducible promoter. Other methods of introducing DNA into cells include the use of liposomes, lipofection, electroporation, a particle gun, or by direct DNA injection.

Conventional recombinant DNA techniques are used in the methods of the disclosure. For example, conventional recombinant DNA techniques are used to introduce the desired polynucleotide (e.g., KLF4, OCT4, SOX2, c-MYC or n-MYC, NANOG or any combination thereof) into differentiated cells to de-differentiate the cells into stem cells. The precise method used to introduce a polynucleotide is not critical to the disclosure. For example, physical methods for the introduction of polynucleotides into cells include microinjection and electroporation. Chemical methods such as co-precipitation with calcium phosphate and incorporation of polynucleotides into liposomes are also standard methods of introducing polynucleotides into mammalian cells. For example, DNA or RNA can be introduced using standard vectors, such as those derived from murine and avian retroviruses (see, e.g., Gluzman et al., 1988, Viral Vectors, Cold Spring Harbor Laboratory, Cold Spring Harbor, N.Y.). Standard recombinant molecular biology methods are well known in the art (see, e.g., Ausubel et al., 1989, Current Protocols in Molecular Biology, John Wiley & Sons, New York), and viral vectors for gene therapy have been developed and successfully used clinically (Rosenberg, et al., 1990, N. Engl. J. Med, 323:370). Other methods, such as naked polynucleotide uptake from a matrix coated with DNA are also encompassed by the disclosure (see, for example, U.S. Pat. No. 5,962,427, which is incorporated herein by reference).

Somatic cells, such as fibroblasts, are transformed or transfected with a polynucleotide encoding a de-differentiation factor(s), e.g., DNA, controlled by or in operative association with one or more appropriate expression control elements such as promoter or enhancer sequences, transcription terminators, polyadenylation sites, among others, and may further include a detectable marker. Following the introduction of the foreign DNA, engineered cells may be allowed to grow in enriched media and then switched to selective media.

Any promoter may be used to drive the expression of the inserted gene. For example, viral promoters include but are not limited to the CMV promoter/enhancer, SV40, papillomavirus, Epstein-Barr virus, elastin gene promoter and beta-globin. The control elements used to control expression of the polynucleotide encoding a de-differentiation factor should allow for the regulated expression of the polynucleotide. If transient expression is desired, constitutive promoters are used in a non-integrating and/or replication-defective vector. Alternatively, inducible promoters could be used to drive the expression of the inserted gene when necessary. Inducible promoters can be built into integrating and/or replicating vectors. For example, inducible promoters include, but are not limited to, metallothionien and heat shock protein.

For example, the de-differentiation factors set forth herein can be cloned into an expression vector (e.g., a retroviral vector such as a pMX retroviral vector). The expression vector can be transformed into a cell of interest (e.g., a fibroblast). A de-differentiation factor can be introduced by transfection or transduction into a somatic cell or stromal cell using a vector, such as an integrating- or non-integrating vector. After introduction, the DNA segment(s) encoding the de-differentiation factor(s) can be located extra-chromosomally (e.g., on an episomal plasmid) or stably integrated into cellular chromosome(s).

Where a retroviral vector is used a virus particle can be generated in a host cell to obtain infectious viral particles (e.g., in cell such as Phoenix-A cells). A cell-type of interest (e.g., a cell type to be de-differentiated) can then be infected with virus and cultured appropriately to select and grow the de-differentiated phenotype. For example, in one embodiment, human dermal fibroblasts are infected twice over 3 days at passage 6 and then re-plated four days later onto a feeder lay (e.g., an irradiated murine fibroblasts feeder layer). In one embodiment, the human dermal fibroblasts are obtained from a subject having an RTT-associated mutation.

The vector can include a single DNA segment encoding a single de-differentiation factor or a plurality of de-differentiation factors in any order so long as that they are operably expressed and function in a recombinant host cell. Where a vector includes one or some of the de-differentiation factors, but not all, a plurality of vectors (e.g., 2, 3, 4, or 5) can be introduced into a single somatic or stromal cell. A marker such as an expressed marker (e.g., a fluorescent protein such as GFP) can be used in combination with the de-differentiation factor to measure expression from the vector. For example, a GFP marker can be used to measure expression from a retroviral vector. The disclosure demonstrates that loss of expression from a retroviral vector comprising a de-differentiation factor can be used to select/enrich for stem cells. Alternatively, one or more markers of de-differentiation (e.g., pluripotent status) can be measured.

The vectors described herein can be constructed and engineered using art-recognized techniques to increase their safety for use in therapy and to include suitable expression elements and therapeutic genes. Standard techniques for the construction of expression vectors suitable for use as described herein are well-known to one of ordinary skill in the art and can be found in such publications such as Sambrook J, et al., “Molecular cloning: a laboratory manual,” (3rd ed. Cold Spring Harbor Press, Cold Spring Harbor, N.Y. 2001), incorporated herein by reference as if set forth in its entirety.

The somatic cells used to obtain induced pluripotent stem cells can be isolated from any number of various tissues of the body. For example, the sample of cells may be obtained from bone marrow, fetal tissue (e.g., fetal liver tissue), peripheral blood, umbilical cord blood, pancreas, skin and the like. In one embodiment, the cells are fibroblasts and in a further embodiment, the cells are obtained from a subject having, or suspected of having, a mutation that causes a neuronal disease or disorder (e.g., causing RTT syndrome or any other neurological disease or disorder). As is well known a somatic cell includes the genetic makeup of the individual and thus any induced pluripotent stem cell obtained from the somatic cell will include the same genetic makeup (e.g., same mutations found in the somatic cells obtained from the subject).

The methods of the disclosure may be applied to a procedure wherein differentiated (lineage committed) cells are removed from a subject, de-differentiated in culture, manipulated to redifferentiate along a specific differentiation pathway (e.g., neuronal cells) and then cultured and studied to (a) determine a mutations phenotypic result and/or (b) screen agents for their effect on the mutant dedifferentiated neuronal cell.

For example, fibroblasts can be removed from a subject, de-differentiated using de-differentiation factors (e.g., with a KLF4, OCT4, SOX2, c-MYC or n-MYC, NANOG agonists or any combination thereof) and optionally mitotically expanded and then redifferentiated (e.g., with a KLF4, OCT4, SOX2, c-MYC or n-MYC, NANOG antagonists or any combination thereof) or factors (including physical stimuli) known to cause differentiation of hESCs down a lineage committed path. In one embodiment, the method comprises removing differentiated cells from an injured or diseased subject. Cells de-differentiated from cells harvested from a subject can then be studied to determine a suitable therapy and/or screened to identify drug/biological candidates of interest to treat the disease or disorder. In one embodiment, the “de-differentiated cells” are differentiated down a lineage committed path to study a particular disease. For example, the de-differentiated cells (e.g., iPSC) can be differentiated down a neuronal lineage to obtain, e.g., astrocytes.

The isolation of cells, such as fibroblasts, from a subject are known. For example, the isolation of fibroblasts may, for example, be carried out as follows: fresh tissue, e.g., a biopsy, samples are thoroughly washed and minced in Hanks balanced salt solution (HBSS) in order to remove serum. The minced tissue is incubated from 1-12 hours in a freshly prepared solution of a dissociating enzyme such as trypsin. After such incubation, the dissociated cells are suspended, pelleted by centrifugation and plated onto culture dishes. Fibroblasts cells will attach to the culture dish before other cells, therefore, appropriate stromal cells can be selectively isolated and grown.

Somatic cell (e.g., a population of somatic cells such as fibroblasts) are obtained from a subject are de-differentiated into induced pluripotent stem cells (iPSCs). For mutations having effects on neuronal processing and development, the iPSCs are then differentiated to neuronal cells. Because the genome of the iPSCs will carry the mutant gene, the differentiated neuronal cells will also carry the same mutation. In this way, the effect of the mutation on neuronal function can be studied. In addition, various factors or agents can be used to modulate the effect of the mutation on neuronal cell function, which may further be specific for the subject that was the source of the cells. In other words, the differentiated neuronal cells can be used to screen agents for effects on the biological function of the mutant neuronal cells. In this way, agents that show a beneficial effect on a particular mutation can then be advanced as potential therapeutics.

In one embodiment, a pluripotent stem cell (e.g., an iPSC) is differentiated into a neural progenitor cell (NPC) using a dual SMAD inhibitor molecules. Exemplary embodiments include differentiating iPSC in the presence of Noggin (e.g., human Noggin polypeptide, such as NP005441.1 or the mature polypeptide contained therein) and SB431542 (collectively, “dual SMAD inhibitors”). Alternative factors (individually and/or in combination) could be used in the disclosed methods in place of either or both of the dual SMAD inhibitors, and/or be used in addition to one or both of these factors. Though these factors are sometimes referred to as “dual” SMAD inhibitors, more or fewer than two factors may be utilized within the scope of these methods. Other dual SMAD inhibitors are known such as, but not limited to, dorsomorphin.

Noggin is a secreted BMP inhibitor that reportedly binds BMP2, BMP4, and BMP7 with high affinity to block TGFbeta family activity. SB431542 is a small molecule that reportedly inhibits TGFbeta/Activin/Nodal by blocking phosphorylation of ACTRIB, TGFbetaR1, and ACTRIC receptors. SB431542 is thought to destabilize the Activin- and Nanog-mediated pluripotency network as well as suppress BMP induced trophoblast, mesoderm, and endodermal cell fates by blocking endogenous Activin and BMP signals. It is expected that agents having one or more of the aforementioned activities could replace or augment the functions of one or both of Noggin and SB431542, e.g., as they are used in the context of the disclosed methods. For example, it is envisioned that the protein Noggin and/or the small molecule SB4312542 could be replaced or augmented by one or more inhibitors that affect any or all of the following three target areas: 1) preventing the binding of the ligand to the receptor; 2) blocking activation of receptor (e.g., dorsomorphin), and 3) inhibition of SMAD intracellular proteins/transcription factors. Exemplary potentially suitable factors include the natural secreted BMP inhibitors Chordin (which blocks BMP4) and Follistatin (which blocks Activin), as well as analogs or mimetics thereof. Additional exemplary factors that may mimic the effect of Noggin include use of dominant negative receptors or blocking antibodies that would sequester BMP2, BMP4, and/or BMP7. Additionally, with respect to blocking receptor phosphorylation, dorsomorphin (or Compound C) has been reported to have similar effects on stem cells. Inhibition of SMAD proteins may also be affected using soluble inhibitors such as SIS3 (6,7-Dimethoxy-2-((2E)-3-(1-methyl-2-phenyl-1H-pyrrolo[2,3-b]pyridin-3-yl-1-prop-2-enoyl))-1,2,3,4-tetrahydroisoquinoline, Specific Inhibitor of Smad3, SIS3), overexpression of one or more of the inhibitor SMADs (e.g., SMAD6, SMAD7, SMAD 10) or RNAi for one of the receptor SMADs (SMAD1, SMAD2, SMAD3, SMAD5, SMAD8/9). Another combination of factors expected to be suitable for generating neural progenitors comprises a cocktail of Leukemia Inhibitory Factor (LIF), GSK3 inhibitor (CHIR 99021), Compound E (gamma secretase inhibitor XXI) and the TGFbeta inhibitor SB431542 which has been previously shown to be efficacious for generating neural crest stem cells (Li et al., Proc Natl Acad Sci USA. 2011 May 17; 108(20):8299-304). Additional exemplary factors may include derivatives of SB431542, e.g., molecules that include one or more added or different substituents, analogous functional groups, etc. and that have a similar inhibitory effect on one or more SMAD proteins. Suitable factors or combinations of factors may be identified, for example, by contacting iPSCs with said factor(s) and monitoring for adoption of neural crest stem cell phenotypes or embryoid bodies, such as characteristic gene expression.

Following differentiation, the NPCs are isolated and collected for further processing. For example, the NPCs can be manually picked from lightly dissociated embryoid bodies (EBs) and placed in suspension (e.g., for about 5 days). The resulting NPCs are grown to confluence and then are incubated with dPBS for 2-10 minutes and then scraped and cultured to form neurospheres using neural growth factor (NGF). The neurospheres are then dissociated and replated and cultured (e.g., with constant shaking). Once the neurospheres are well formed, ROCK inhibitor or similar agent can be added, with the elimination of FGF from the media, and cultured for 1 to 3 days. The cells are then cultured without ROCK inhibitor in NG media without FGF for about 1 week. Astrocyte media is then added to the neurospheres for about 2 weeks and cultured while shaking at about 90 rpms. After about 2 weeks the spheres are plated and astrocytes project outside of the spheres to populate the plate surface.

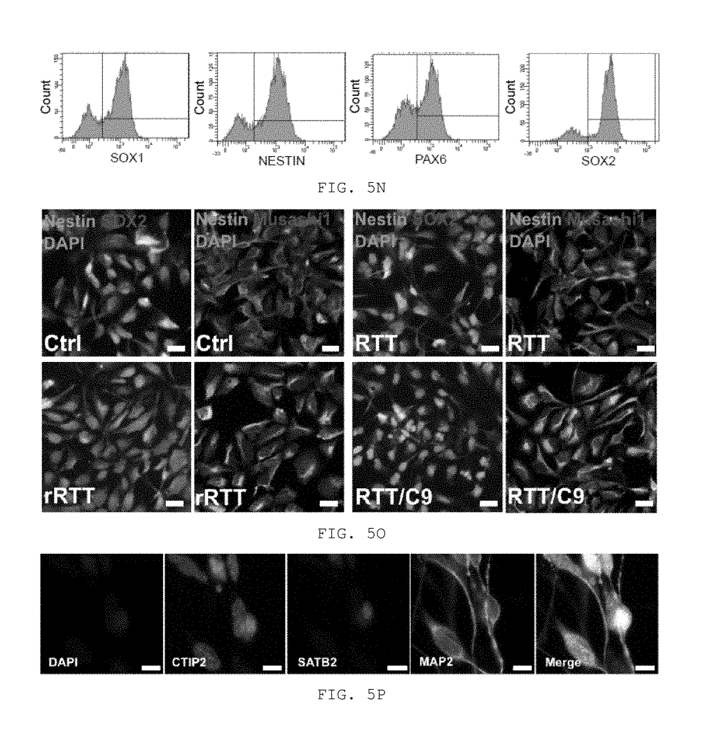

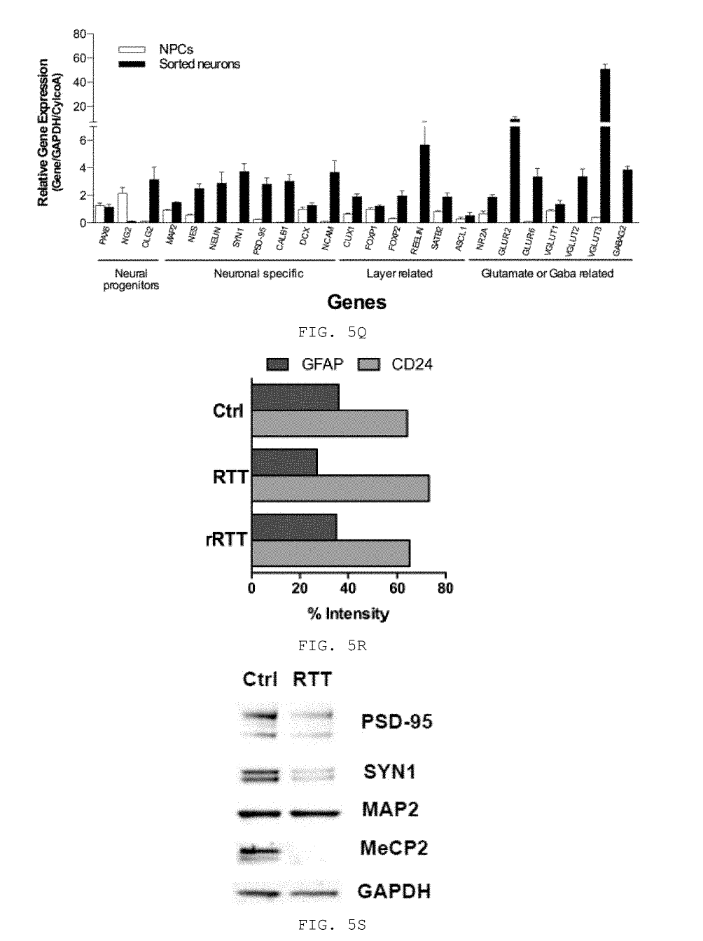

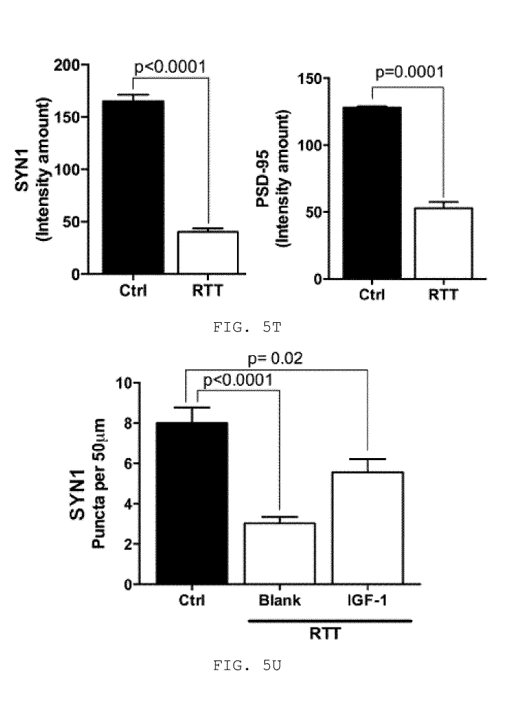

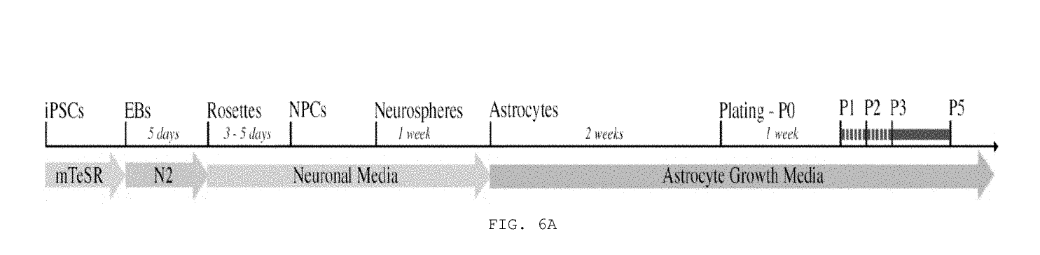

FIG. 6A depicts a general method of the disclosure. For example, iPSCs are differentiated into neural progenitor cells (NPCs) using dual SMAD inhibitor molecules, their isolation and collection is through manual rosette picking from lightly dissociated embryoid bodies (EBs) placed in suspension for five days. NPCs from a confluent 100 mm diameter plate are incubated with dPBS (Mediatech, VWR) at 37° C. for 5 minutes and then scraped to form neurospheres using 9.5 mL of NGF. Cells are gently dissociated by pipetting and distributed in 3 mL per well in a six-well plate and kept at constant shaking (90 revolutions per minute (rpm)). Media is changed on the day after cells are suspended or once the neurospheres are well formed. Rock inhibitor or similar agent is then added to a final concentration of 5 μM for 48 hours concomitant with the removal of FGF from the media in the next media change. After the removal of Rock inhibitor, NG media without FGF is used for about a one week culture. Astrocyte growth media (Lonza) is then added to the spheres for two weeks under shaking at 90 rpm. After two weeks the spheres are plated and astrocytes project outside of the spheres to populate the plate surface.

It should also be noted that the iPSCs and NPC and astrocytes obtained herein can be cultured, expanded and used as cell banks. For example, once human induced pluripotent stem cells, NPCs or astrocytes have been established in culture, as described herein, they may be maintained or stored in cell “banks” comprising either continuous in vitro cultures of cells requiring regular transfer or cells which have been cryopreserved.

Cryopreservation of stem cells, or other cell of the disclosure, may be carried out according to known methods, such as those described in Doyle et al., (eds.), 1995, Cell & Tissue Culture: Laboratory Procedures, John Wiley & Sons, Chichester. For example, but not by way of limitation, cells may be suspended in a “freeze medium” such as, for example, culture medium further comprising 15-20% fetal bovine serum (FBS) and 10% dimethylsulfoxide (DMSO), with or without 5-10% glycerol, at a density, for example, of about 4-10×10⁶ cells/ml. The cells are dispensed into glass or plastic vials which are then sealed and transferred to a freezing chamber of a programmable or passive freezer. The optimal rate of freezing may be determined empirically. For example, a freezing program that gives a change in temperature of −1° C./min through the heat of fusion may be used. Once vials containing the cells have reached −80° C., they are transferred to a liquid nitrogen storage area. Cryopreserved cells can be stored for a period of years, though they should be checked at least every 5 years for maintenance of viability.

The cryopreserved cells of the disclosure constitute a bank of cells, portions of which can be withdrawn by thawing and then used to produce a cell culture comprising stem cells, NPCs or astrocytes as needed. Thawing should generally be carried out rapidly, for example, by transferring a vial from liquid nitrogen to a 37° C. water bath. The thawed contents of the vial should be immediately transferred under sterile conditions to a culture vessel containing an appropriate medium. It is advisable that the cells in the culture medium be adjusted to an initial density of about 1-3×10⁵ cells/ml. Once in culture, the cells may be examined daily, for example, with an inverted microscope to detect cell proliferation, and subcultured as soon as they reach an appropriate density.

In addition, the cells of the disclosure can be used, for example, to screen in vitro for the efficacy and/or cytotoxicity of compounds, growth/regulatory factors, pharmaceutical compounds, and the like on the stem cells or a particular lineage of cells derived/differentiated from the stem cells, to elucidate the mechanism of certain diseases by determining changes in the biological activity.

### EXAMPLES

iPSC Generation.

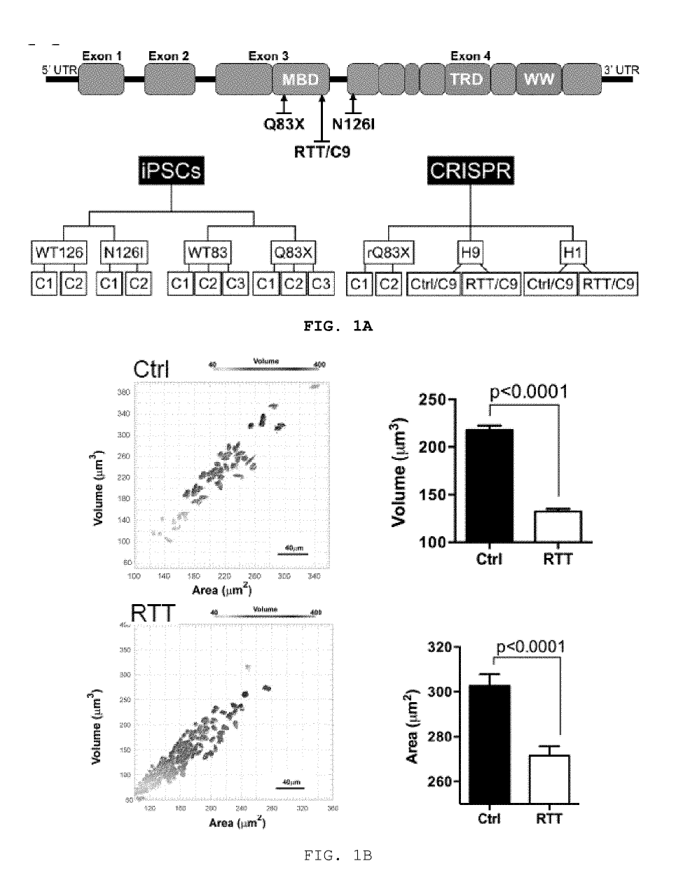

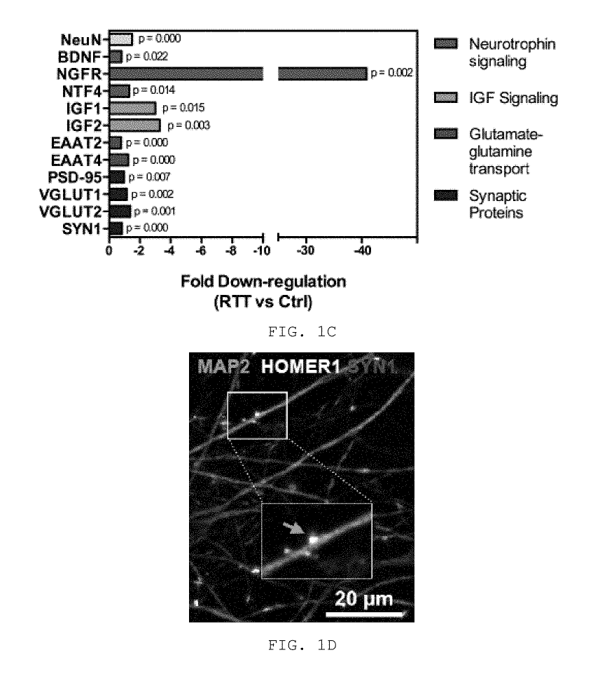

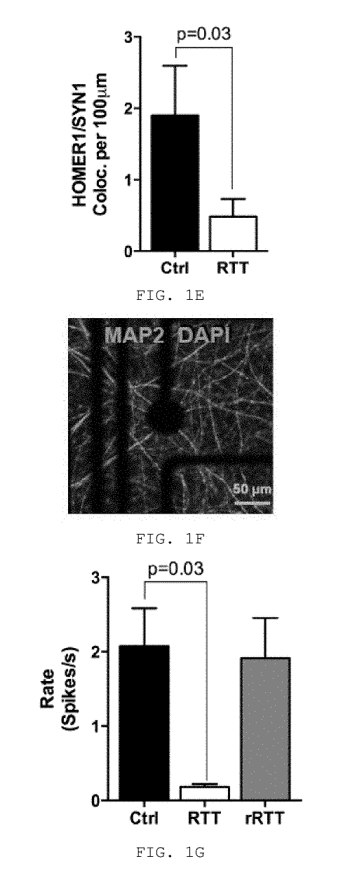

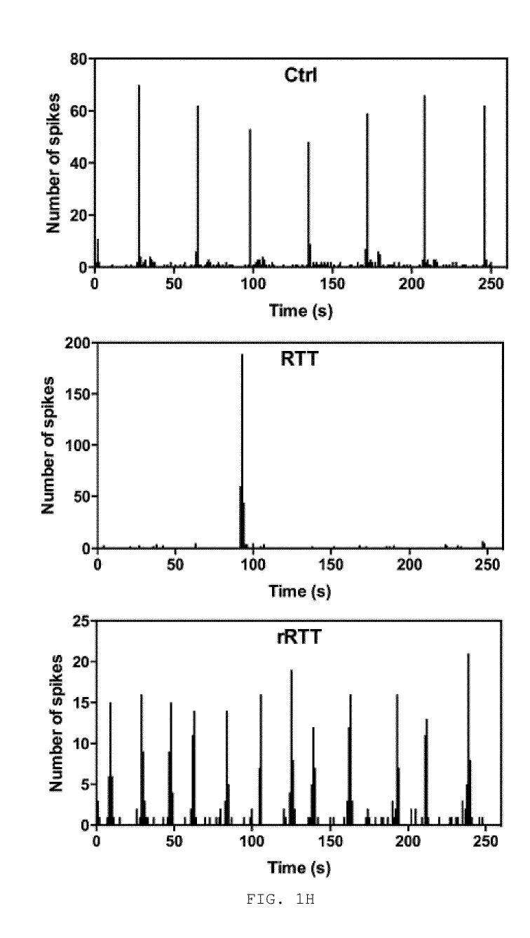

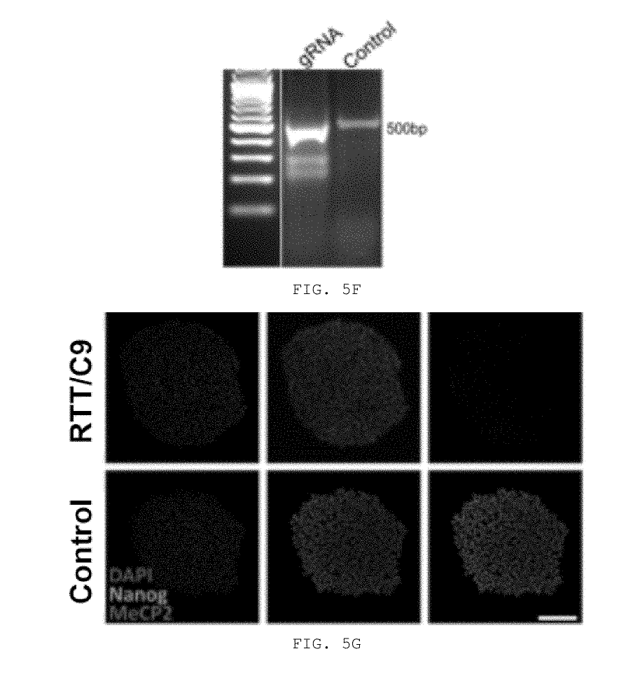

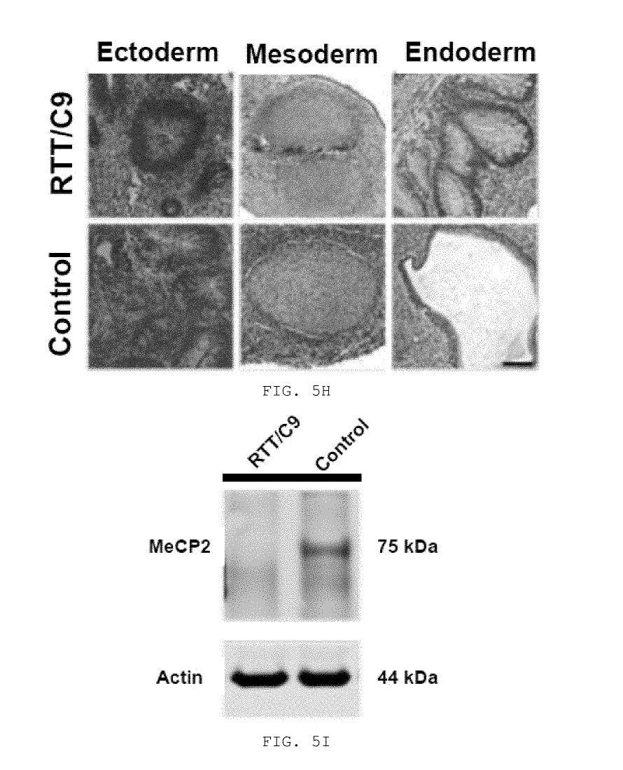

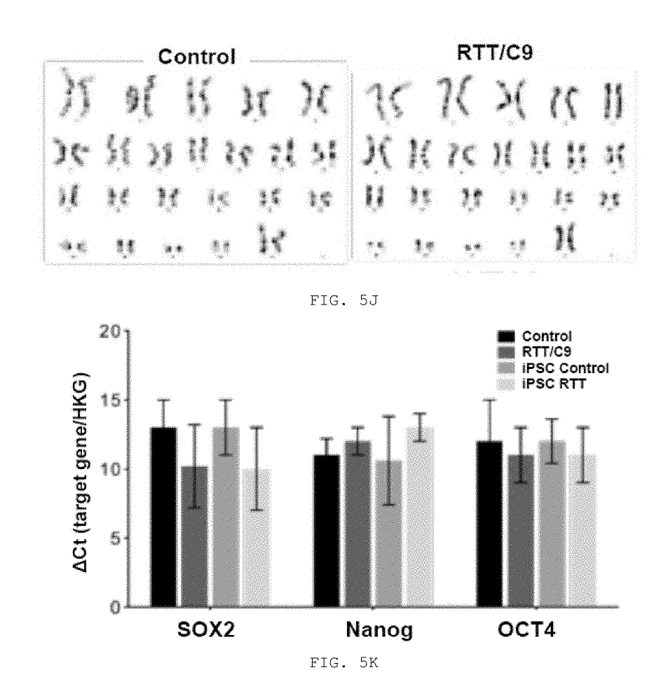

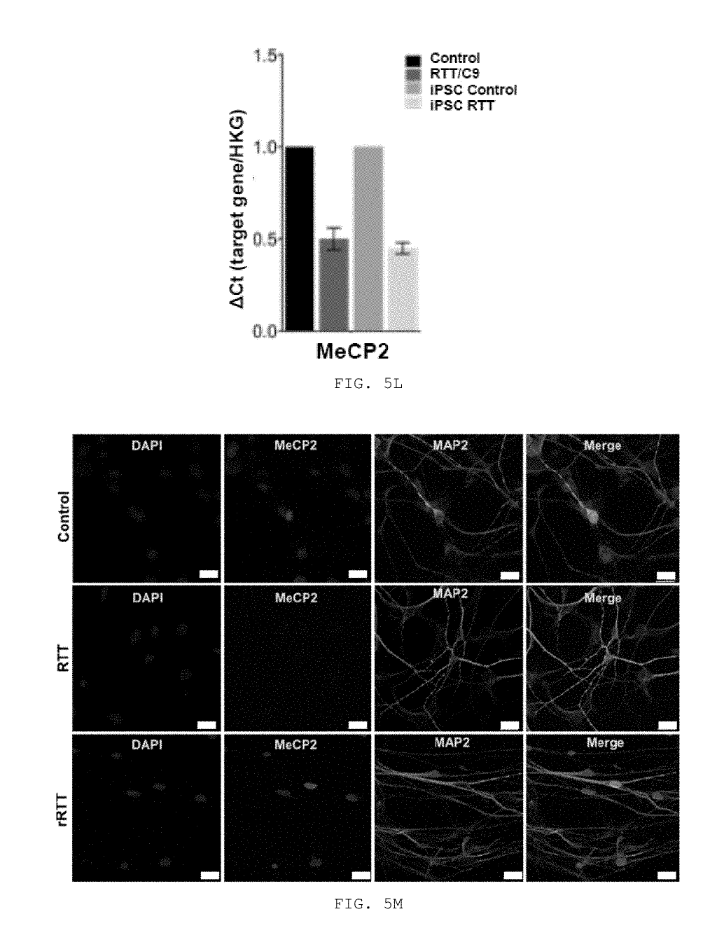

Male fibroblasts carrying two distinct MeCP2 mutations (FIG. 1A; FIGS. 5A-C, Table 1) and two controls (from their respective non-affected fathers) were obtained from explants of dermal biopsies. Retrovirus transduction (containing OCT4, SOX2, KFL4 and MYC) and iPSC generation were performed as previously described (Marchetto et al., 2010; Nageshappa et al., 2015). All experiments were performed with a minimum of two clones per patient cell line, repeated three times (for details please see Table 4). All cell lines were genotyped for SNPs and chromosomal aberrations, where any line that presented any abnormality was discarded from this study.

TABLE 1
MeCP2 mutations, phenotype description and
respective cell-lines used in this study.

| | | MeCP2 | Nucleotide | |
|---|---|---|---|---|
| Cell line name | Gender | mutation | change | Individual phenotype |
| WT83 | Male | WT | — | Healthy individual. The |
| | | | | karyotype is 46, XY; normal |
| | | | | diploid male |
| WT126 | Male | WT | — | Healthy individual. The |
| | | | | karyotype is 46, XY; normal |
| | | | | diploid male |
| Q83X | Male | de novo | 247C > T | Respiratory difficulties and |
| | | | | seizures from birth. Inability to |
| | | | | swallow or suck, being tube |
| | | | | fed. Apnea from birth and |
| | | | | dependent of a ventilator for |
| | | | | breathing. Increased muscle |
| | | | | tone in extremities, |
| | | | | constipation, diarrhea and |
| | | | | gastroesophageal reflux. Loss |
| | | | | of acquired motor skills and |
| | | | | language. Growth retardation |
| | | | | and scoliosis. The karyotype is |
| | | | | 46, XY; affected diploid male |
| N126I | Male | de novo | c.377A > T | Significant developmental |
| | | | | delay. Loss of motor skills. |
| | | | | Periodic breathing with |
| | | | | prominent apnea and |
| | | | | hyperventilation at age one |
| | | | | month. Persistent drooling, |
| | | | | bruxism, gastroesophageal |
| | | | | reflux and constipation. Hand |
| | | | | sterotypies. Muscle loss and |
| | | | | prominent dystonia at angles. |
| | | | | Growth retardation and |
| | | | | scoliosis. The karyotype is 46, |
| | | | | XY; affected diploid male. |

Isogenic Cell Line Generation.

Using human embryonic cell lines (hESCs) H1 and H9 additional amino acids were incorporated in order to insert frame-shift mutations in the MeCP2 gene. This incorporation resulted in the creation of early stop codons rendering a non-functional MeCP2 protein (cell lines used: H1, H9, 184, 906 and 904). Mutagenesis as well as off-targets, were confirmed by exome sequencing techniques (Tables 2). None of the predicted off-targets were mutagenized in any of the clonally derived cell lines (Table 3). Comparing the cell line exomes against the parental H1/H9 exome, several homozygous InDels in each line were identified that would lead to a frame-shift mutation, including at the expected position in the MeCP2 locus in the H1 and H9 lines. The sequence of each InDel was analyzed and found that none of the sequences of these homozygous InDels matched sequence to the guide RNA. Therefore, it was concluded that each nonspecific InDel already existed in subpopulations in the parental H1/H9 hESCs and arose during clonal expansion. The off-target analysis data are consistent with previous findings (Tables 2 and 3) (Smith et al.; Veres and Talkowki). Protein detection was confirmed by immunofluorescence using anti-MeCP2 antibodies (Diagenodes) and western blot techniques. The rescue isogenic cell line from Q83X patient converted the early stop codon at the amino acid in position 83 into a Glycine, resulting in a functional MeCP2 protein, with expression confirmed by Immunofluorescence (FIG. 5M).

TABLE 2
Exome sequencing results from isogenic cells
related to FIG. 1A and FIGS. 5D-K.

| Sample | Coverage | Control | Deletion |
|---|---|---|---|
| Control | 117 | 117 | 0 |
| RTT/C9 | 161 | 0 | 161 |

TABLE 3
Control versus RTT/C9 CRISPR/Cas9 off-target genes
related to FIG. 1A and FIGS. 5D-K.

| Chromosome | Position | Reference | Mutation | Gene |
|---|---|---|---|---|
| 5 | 76373240 | A | G | ZBED3 |
| 14 | 105414790 | A | G | AHNK2 |
| 11 | 56853585 | C | T | TRIM5 |
| 4 | 22438154 | C | A | GPR125 |
| 17 | 38975328 | G | A | KRT10 |

Neuronal Differentiation.

Neural progenitor cells (NPCs) were generated through embryoid bodies. The NPC population was expanded using the NGF media comprising DMEM/F12 media supplemented with 0.5×N2, 0.5× Gem21 supplement (Gemini Bio-products), 20 ng/mL of FGF and 1% penicillin/streptomycin. To differentiate the NPCs into neurons, a 70% confluent plate was treated with 5 μM of ROCK inhibitor for 48 hrs (Y-27632, Calbiochem) in the absence of FGF, with regular media changes every 3 or 4 days.

Neuronal Nuclei Dimension Characterization.

NPCs were seeded, differentiated into neurons, fixed and stained with DAPI after 4 weeks of differentiation. Z-stack confocal images from MAP2 and SYN1 labeled neurons. Nuclei reconstruction was made using the powerful volumetric tool in the Imaris Software (Bitplane). Extracted volume and area data were plotted using Vintage tool also using the Imaris Software.

Neuronal Puncta Quantification.

NPCs were differentiated, as described, into neuronal cells. After 4 weeks in culture, cells were fixed and stained for SYN1, Homerl or PSD-95 along with MAP2 for cell identification. Images were taken at 63× magnification in a Z-stack experiment using a Zeiss Apotome microscope. Puncta was identified and calculated using the Imaris software (Bitplane). At least 12 images of the same slide were taken and processed, each slide performed in triplicate. In cell western analysis were also used to quantify PSD-95 and SYN1 levels using the Odissey machine (LiCor).

Multi-Electrode Array Readings (MEA).

Experiments were carried out based on seeding NPCs in a MEA plates using the Maestro machine (Axion BioSystems) on a 12-well plate. Each well was seeded with ten thousand NPCs in triplicate for 12 wells. For MEA experiments, neuronal differentiation was performed as described herein, with the NGF media being replaced for Neurobasal A media (Life Technologies) on their second week of differentiation. Each well was coated with poly-L-ornithine and laminin prior cell seeding. Cells were fed twice a week and measurements were taken before the media was changed.

Generation of Human iPSC-Derived Astrocytes and Primary Astrocyte Culture.

NPCs from a confluent 100 mm diameter plate were incubated with dPBS (Mediatech, VWR) at 37° C. for 5 minutes and then scraped to form neurospheres using 9.5 mL of NGF. Cells are gently dissociated by pipetting and distributed in 3 mL per well in a six-well plate and kept at constant shaking (90 revolutions per minute (rpm)). Media was changed on the day after cells were suspended or once the neurospheres were well formed (FIGS. 6A-D). Rock inhibitor was added to a final concentration of 5 μM for 48 hours concomitant with the removal of FGF from the media in the next media change. After the removal of Rock inhibitor, NG media without FGF was used for one week. Next, astrocyte growth media (Lonza) was added to the spheres for two weeks still shaking at 90 rpm's. After two weeks the spheres are plated and astrocytes project outside of the spheres to populate the plate surface.

Fluorescence Measurements of Intracellular Cat.

The coverslip containing the cell was attached to the bottom of a special chamber for microscopy applications (Warner Instrument, Hamden, Conn.). Cells were loaded with 2 μM calcium-sensitive fluorescent dye Fluo 4-AM (Molecular Probes, Eugene, Oreg.) for 40 min in the dark at 37° C. After rinsed with Hepes buffer and incubated for an additional 10 min, the cells were ready for calcium imaging experiments. HEPES buffer contained (in mmol/L): NaCl 140, KCl 5, CaCl₂ 2, MgCl₂ 2, HEPES 10, Glucose 10 (315 mOsm, pH 7.4). The chamber was secured on the stage of an inverted microscope equipped with fluorescent imaging system (Zeiss Axiovert 200M microscope, Zeiss).

Co-Culture Experiments.

Astrocytes and neurons were differentiated in parallel from NPCs in separate plates. After astrocytes are characterized by immunostaining, live cells were plated at 5×10⁶ per well in a 24-well plate containing glass coverslips until 80% of confluence is achieved. Neuronal enrichment is achieved by removal of CD44 and CD184 positive cell population in the mixed neuronal population using magnetic sorting and plated on top of astrocytes. A total of 2×10⁴ neurons are plated in each of 24 wells pre-coated with astrocytes and kept for two weeks in astrocyte media before fixation. Cells were fixed and stained as described herein.

Quantification of Neuronal Morphology.

Analysis of neuronal morphology was performed using the Neurolucida software (MBF Bioscience, Williston, Vt.). Only mature neurons with visible dendritic spines were selected for the analysis. From each experimental condition, i.e., control neurons plated with control astrocytes, control plated with RTT neurons, RTT plated with RTT astrocytes, and RTT plated with control astrocytes, ten cells per experimental condition were randomly chosen for the analysis, resulting in a total of 200 cells traced. Dendrites/spines were quantified along x-, y-, and z-coordinates using ‘Live Image’ option on Neurolucida software v. 10 (MBF Bioscience, Williston, Vt.) interfaced with a Nikon Eclipse E600 under 40× magnification with a 40×/1.30 oil objective.

IL-6 Media Levels by Flow.

Astrocytes were seeded in 96 well plates with 10,000 cells per well. Once cells reach full confluence of the well, media was changed and incubated for 48 hours prior collection and stored at −80° C. for further processing. Cytometric Bead Array (CBA) kit for human IL-6 (BD Biosciences) was performed according to manufacturer's instructions and ran in a 96 well plate using a FACS Canto 2 instrument (BD Biosciences).

Multiplex ELISA and Cytokine Expression Array.

Human TH1/TH2 10-Plex multi-spot 96 well with 10 spot plate was used for the ELISA validation of secreted cytokines (MSD, Meso Scale Discovery cat n. N05010B-1). The assay is pre-made to measure human IFN-γ, IL1-β, IL-2, IL-4, IL-5, IL-8, IL-10, IL-12p70, IL-13 and TNF-α from cultured media. Cells were plated at a confluence of 0.05×10⁶, had their media changed 24 hours and the fresh media was collected 48 hours to be assayed using the MSD. Values were normalized with media added to the plate in the absence of any cell type.

Statistics.

The number of clones and experimental repeats from each method used in this work can be found in Table 4. The graphs were made and analyzed using the GraphPad Software Prism 6. All samples from all neuronal quantification experiments were analyzed to assess Gaussian distribution, and they all were positive in this analysis. When only one hypothesis was tested, Control and RTT were compared using a non-parametric Student's t-test, unpaired with Welch's correction. When multiple hypotheses were present, a one-way ANOVA (nonparametric) was performed with multiple comparisons, compared always to WT, with Bonferroni correction. Error bars are based in the standard error of the mean (SEM) of each sample.

TABLE 4
Number of clones from iPSCs used in each experiment in this study. The
number indicates the times a clone was used for that particular method.

| | Subjects | | | | | | | | | | | | | |
|---|---|---|---|---|---|---|---|---|---|---|---|---|---|---|
| | WT83 | | WT126 | | Q83X | | N126I | | | | | | | |
| | Clones | | | | | | | | Isogenic lines | | | | | |
| Experiments | C1 | C2 | C1 | C2 | C1 | C2 | C1 | C2 | rRTT | | RTT/C9 | | Ctrl/C9 | |
| Pluripotency assays | 2 | | 2 | | 2 | | 2 | | 1 | 1 | 1 | 1 | 1 | 1 |
| RNAseq | 1 | | 1 | | 1 | 1 | 1 | 1 | 1 | 1 | 1 | 1 | 1 | 1 |
| Karyotyping G-Band | 1 | | 1 | | 1 | | 1 | | 1 | | 1 | | 1 | |
| DNA Bead Array | 1 | 1 | 1 | 1 | 1 | 1 | 1 | 1 | 1 | 1 | 1 | 1 | 1 | 1 |
| NPCs FACS | 1 | 1 | 1 | 1 | 1 | 1 | 1 | 1 | 1 | | 1 | | 1 | |
| Nuclei Volume/Area expt. | 3 | 3 | | | 3 | 3 | | | | | | | | |
| MEA | 3 | 3 | | | 3 | 3 | | | 3 | 3 | 3 | 3 | 3 | 3 |
| IL-6 Flow media levels | 3 | 3 | 3 | | 3 | 3 | 3 | | 3 | 3 | 3 | 3 | 3 | 3 |
| Astrocyte differentiation | 3 | 3 | 3 | 3 | 3 | 3 | 3 | 3 | 3 | 3 | 3 | 3 | 3 | 3 |
| Astrocyte FACS | 3 | 3 | 3 | | 3 | | 3 | | 2 | 2 | 2 | 2 | 2 | 2 |
| Calcium wave assay | 3 | | 2 | | 2 | | 2 | | | | | | | |
| qPCR expression | 3 | 3 | 3 | 3 | 3 | 3 | 3 | 3 | 3 | 1 | 3 | 1 | 3 | 1 |
| Co-culture model | 3 | | 3 | | 3 | | 3 | | | | | | | |

Biological Data Accession Codes.

Datasets are available under restricted access at NCBI BioProject accession code: PRJNA297130.

Immunofluorescence.

Cell media was aspirated and cells were washed once with dPBS (Mediatech, VWR). Fixation was performed using 4% paraformaldehyde diluted in dPBS (Corning) for 15 minutes at room temperature. Cells were then washed three times with dPBS and permeabilized with 0.1% Triton X-100 (Promega) diluted in dPBS for 15 minutes. Fixed cells were blocked with 2% BSA (Gemini) overnight and then incubated overnight with the primary antibody at 4° C. In the following day, cells were washed 3 times with dPBS and blocked again for one hour with 2% BSA at room temperature. Secondary antibodies were added following the blocking solution and kept for one hour at room temperature. Cells were washed three times with dPBS and DAPI was added to the fixed cells for 5 minutes at room temperature. Cells were washed once after the DAPI addition with dPBS and mounted in slides using Pro-long Gold antifade reagent (Invitrogen). Antibodies are described below.

Antibodies Used in this Project:

rat anti-CTIP2 (Abcam); anti-GFAP: chicken (Abcam), rabbit (Dako), mouse (Millipore); mouse anti-Homerl (Synaptic Systems); goat anti-Lin28 (R&D Systems); anti-MAP2 rabbit (Millipore), chicken (abcam); rabbit anti-MeCP2 (Diagenode); rabbit anti-Musashil (Abcam); goat anti-Nanog (R&D Systems); mouse anti-Nestin (Abcam); mouse anti-NeuN (Millipore); mouse anti-OCT4 (Santa Cruz); rabbit anti-SATB2 (abcam); rabbit anti-S100β (abcam); rabbit anti-SOX2 (cell signaling); rabbit anti-Synapsinl (Millipore); mouse anti-actin (Abcam). The secondary antibodies: Alexa Fluor 488, Alexa Fluor 555 and Alexa Fluor 647 (Life Technologies).

Karyotyping.

G-banding karyotype was performed at Molecular Diagnostic Services, Inc. (San Diego, Calif.). Cells were provided at subconfluent stage. This procedure was repeated every 10 passages of the iPSC and cell lines with abnormal karyotypes were discarded. In addition to G-banding karyotyping, two clones of each cell line had 200 ng of their DNA assessed for copy-number variation (CNV) and single-nucleotide polymorphisms using the Illumina Infinium Human Core Exome Bead Chip following manufacturer's instructions at IGM Genomics Core at UCSD. Illumina Genome Studio V2011.1 with the Genotyping module Ver. 1.9.4 was used to normalize data and call genotypes using reference data provided by Illumina. Illumina's cnvPartition and gada R package were used to automatically detect aberrant copy number region. In addition, the B Allele Frequency (BAF) and Log R Ratio (LRR) distributions were manually checked to determine additional CNAs not detected by the software. Sample identification or relatedness was assesses by comparing called genotypes for each sample. The absolute number of different genotypes were counted and the Euclidean distances calculated to identify relatedness of the samples.

HT Sequencing Data Quality Check and Expression Analysis.

To recovery high quality reads from HT sequencing, we first filtered the sequenced libraries by considering bad quality reads and sequence contamination with different types of artifacts, such as sequencing primers and adapters as well as short fragments. The software NGS QC Toolkit (Patel and Jain, 2012) was used for this step. High quality reads for each sequenced library were then aligned against the reference transcriptome from ENSEMBL human database (release 72), which includes all known genes, novel and pseudo-gene predictions. The alignment was based on a non-gapped approach using the software Bowtie2 (Langmead and Salzberg, 2012), with default parameters. For each sequenced library, the alignments were then analyzed by in house software to extract the absolute number of aligned reads for each individual gene (considering all gene isoforms) from the reference ENSEMBL transcriptome. Next, using a read-count approach that is based on a binomial negative distribution, expression of individual transcripts per sample was normalized for gene expression analysis. The expression, normalized based on the library size and aligned reads, was then used for statistical significance analysis. To control false-positive significance gene expression variation, we applied FDR correction (False Discovery Rate), from Benjamini & Hochberg (reviewed in 2001) (Benjamini et al., 2001), over the statistical significance found between the samples. These analyses were performed using DESeq (Anders and Huber, 2010), a Bioconductor R packge. In our analysis, genes were considered as differentially expressed when the statistical significance p-value was less than 0.05.

Gene Details for Microarray Analysis.

Nanog (ENSG00000111704) transcripts: ENST00000541267; ENST00000229307; ENST00000526434; ENST00000526286. TRA-1-60 (ENSG00000128567) transcripts: ENST00000446198; ENST00000484346; ENST00000378555; ENST00000322985; ENST00000490761; ENST00000487965; ENST00000482581; ENST00000465001; ENST00000541194; ENST00000537928. Lin28 (ENSG00000131914) transcripts: ENST00000326279; ENST00000254231. Klf4 (ENSG00000138814) transcript ENST00000512215. DPPA2 (ENSG00000163530) transcript ENST00000478945. GDF3 (ENSG00000184344) transcript ENST00000329913. ESG1 (ENSG00000203909) transcript ENST00000370370. Sox2 (ENSG00000181449) transcripts: ENST00000431565; ENST00000325404. POU5F1 (ENSG00000204531) transcripts: ENST00000259915; ENST00000512818; ENST00000513407; ENST00000441888; ENST00000471529; ENST00000461401.

Sample Clustering.

To perform clustering analysis over RNA-seq samples, the normalized expression values for all of the identified genes per sample were used. These data were combined in an expression matrix having the rows representing a specific gene and the columns representing the expression of that gene on each library. A distance matrix was then created, which represents the Euclidian distance between all the gene expression data between pairs of samples. To create the dendrogram, the distance matrix is then analyzed and, using the single linkage method, samples having smaller distances are clustered together. Additionally, a heatmap was created that is based on the matrix distances. Each value from the matrix, representing a Euclidian distance between two samples, is converted to a color, ranging from green (samples are very different) to red (samples are exact more similar). PCR for cell lineage characterization. iPSCs were induced into EBs and 10% of fetal bovine serum was added to the media inducing cell differentiation into the three germlayers (ectoderm, mesoderm and endoderm). Cells were extracted using Trizol Reagent (Life Technologies, CA) according to the manufacturer's suggestions. Reverse transcriptase was performed using SuperScript III (Invitrogen) according to manufacturer's instructions once DNAse (Invitrogen) treatment was performed. RNA quality was assessed by electrophoresis in a 2% agarose gel and by A260/A280 ratio measured by a nanodrop. Three (3) μg of RNA was used to generate cDNA. The sequences for the primers used here are described on a separate section below.

Teratoma Formation.

Confluent iPSCs colonies were dissociated, re-suspended in PBS containing Matrigel® (1:1) and injected subcutaneously into dorsal flanks of immune compromised mice as previously described (Nageshappa, et al., 2015).

Astrocyte Differentiation Details.

Gentamicin provided with the astrocyte media kit (Lonza) was replaced with 1% penicillin/streptomycin, due to the proven ability of this antibiotic to act as a read-trough drug for stop codon mutations possibly rescuing RTT phenotypes (Marchetto, et al. 2010). Once the astrocytes start to differentiate from progenitor cells, they begin to produce laminin and attach to the bottom of the plate being ready to be plated (FIG. 6B). Cells attached to the bottom of the six wells were discarded and spheres in suspension were plated on a double-coated plate similar to the neuronal differentiation method for cell expansion: 10 μg/mL of poly-ornithine (Sigma) for plastic or 100 μg/mL for glass coverslips. After poly-ornithine coating for 2 hours or overnight at incubator, plates were washed twice with water and coated with laminin (ThermoFisher Scientific), at 2.5 μg/mL for plastic and 5 μg/mL for glass coverslips. When the spheres were plated, the astrocytes grew out of the sphere and spread on the plate to form a multilayer cell formation (FIG. 6C-D). After the first passage, cells surrounding the neurospheres were dissociated enzymatically using accutase (Cellgro) papain (Worthington Biochemical Corp.) and plated, spheres were kept for continuous generation of astrocytes in a parallel dish. For astrocyte-enrichment of the population, neurospheres were removed manually by vacuum suction using a Pasteur pipet and the result was a confluent and homogeneous plate of GFAP- and S100β-expressing astrocytes illustrated in FIG. 6D. Cells can be kept for 4-5 passages or until they reach a senescent stage, which varies according to cell line. Astrocytes used in this study were characterized by immuno-staining procedures and used at passage 3 to 5. Human primary astrocytes labeled Normal Human Astrocytes (NHA) were obtained from Lonza and cultivated according to the manufacturer's instructions.

Protein Isolation and Western Blot Analysis.

Cells were isolated, suspended in RIPA lysis buffer (Upstate) supplemented with 1% protease inhibitor cocktail (Sigma). Twenty micrograms of total protein was separated on 10% gradient SDS-polyacrylamide gel, transferred to a nitrocellulose membrane and probed with a primary antibody against specific proteins, followed by infrared secondary antibodies detected in the Odissey® CLx system as instructed by manufacturer (Li-Cor).

FACS.

Cells were dissociated with accutase (Cellgro), washed with dPBS, filtered using a 70 mm cell strainer, fixed with Cytofix Fixation Buffer, and permeabilized with Perm Buffer III (all from BD Biosciences). Cells were stained with specific antibodies to for 30 minutes and washed. When needed, cells were then stained with anti-rabbit Alexa Fluor 488 (Invitrogen, 1:1,000) or using fluorochrome-conjugated antibodies (BD Biosciences, one test per 1×10⁶ cells) for 30 minutes and washed with stain buffer. FACS analysis was performed on a FACSAria II (BD Biosciences) and analyzed using FlowJo (Tree Star).

Quantitative PCR.

Total RNA was extracted by trizol (Life Technologies) and cDNA was prepared using SuperScript III first-strand kit (Life Technologies). The cDNA obtained from RNA extraction following RT-PCR was subjected to standard quantitative PCR using iQ SYBR green Master Mix (Biorad). Each sample was run in triplicate in a 96-well format CFX machine (Biorad). Primers were designed for specific genes using Primer3 software (v. 0.4.0) or available at the Harvard Primer Bank online. The neuron-specific primers were previously described (Pasca et al., 2011). The PCR cycle conditions were as follows: 2 minutes, 30 seconds at 95° C., 30 seconds at 95° C., 30 seconds at 60° C., and 45 seconds at 72° C. for 50 cycles followed by the melting curve protocol to verify the specificity of amplicon generation. A minimum of two housekeeping genes were used: Cyclophilin A and GAPDH. All remaining quantitative PCR analysis was made by absolute quantification, relative to control of the housekeeping genes using standard curves. A pool consisting of a third of the amount in each sample was gathered to form a standard curve; standard 1 (starting quantity of 1000). Standards 2 to 4 were generated after a 1:5 dilution curve starting with standard 1 to make a relative standard curve. Final expression values were calculated dividing the mean starting quantity (SQ) from our target gene by two housekeeping genes. Details on the 2(−ΔΔCt) method can be found in Nageshappa, et al., 2015.

Astrocyte Calcium Wave Propagation.

Calcium waves were induced by focal mechanical stimulation of single astrocytes in the center of the field of view as described in Scemes et al., 1998. A 175 W Xenon lamp and a wavelength switcher (Lambda DG-4, Sutter Instrument) provided 485 nm fluorescence excitation. The emission from Fluo-4 loaded cells is detected at a wavelength of 520 nm using an F-Fluar 40×/1.3 NA oil immersion objective and an attached 12-bit CCD camera. Changes in Fluo-4 fluorescence intensities emitted at 520 nm were acquired at 0.67 Hz. The calcium imaging data were visualized and analyzed off-line in MetaMorph software (Universal Imaging Corporation). The fluorescent intensity of Fluo-4 was determined in individual cells in every astrocyte soma in the field of view. The distance that calcium waves propagated within certain time was determined by measuring the distance from the stimulating point to the farthest responding cell in the field of view (Scemes et al., 1998).

Magnetic Cell Sorting.

The neuronal population was enriched from a full NPC plate differentiated for 4 weeks into neurons, dissociated using a neurosphere dissociation kit (Miltenyl Biotec) formulated with papain, and sorted using PE-conjugated antibodies against CD184 (BD Biosciences) and CD44 (BD Biosciences) following a second incubation using PE-conjugated with magnetic beads (MACs) markers described in Yuan et al., 2011. Briefly, cells were dissociated using the neurosphere dissociation kit (MACS), incubated with the CD44-PE and CD184-PE (both antibodies were from BD Biosciences) for 15 minutes in the dark at 4° C. and washed twice with dPBS to remove unbound antibodies before sorting. Next the cells were incubated with PE antibody conjugated with magnetic beads (MACS). Following the antibody incubation cells are loaded into a magnetic column and the cells that are CD44 and CD184 positive are retained in the column and the remaining enriched neuronal population is obtained and counted for viability and seeding.

Quantification of Dendritic Morphology.

Since it was recommended that the application of Sholl's concentric spheres or Eayrs' concentric circles for the analysis of neuronal morphology should not be applied when neuronal morphology is analyzed in three dimensions (Uylings et al., 1986), dendritic tree analysis was conducted with the following measurements (FIG. 6H); (1) soma area, representing the area of the cell body, traced at its widest point in the two-dimensional plane; (2) Dendrite number, representing the number of dendritic trees emerging from the cell body; (3) total dendritic length, representing the summed length of all dendrites per neuron and (4) dendritic segment count representing the total number of dendritic segments per neuron. A dendritic segment was defined as part of the dendrite between: (a) two branching points; (b) soma and the first branching (in the case of 1st order segments); or (c) last branching point and the termination of the dendrite. The density of dendritic spines was calculated as the average number of spines per 1 μm of dendritic length. Dendritic segments were assigned orders following centrifugal ordering scheme. The segments closest to the cell body were termed 1st order segments, with segment order increasing after each branching point (2nd order, 3rd order, etc.).

Analysis for MEA Readings.

Recordings were performed using a Maestro MEA system and AxIS software (Axion Biosystems), using a band-pass filter with 10 Hz and 2.5 kHz cutoff frequencies. Spike detection was performed using an adaptive threshold set to 5.5 times the standard deviation of the estimated noise on each electrode. Each plate first rested for five minutes in the Maestro, and then ten minutes of data was recorded in order to calculate the spike rate per well. Multi-electrode data analysis was performed using the Axion Biosystems Neural Metrics Tool, wherein electrodes that detected at least 5 spikes/minute were classified as active electrodes. Bursts were identified in the data recorded from each individual electrode using an adaptive Poisson surprise algorithm. Network bursts were identified for each well, using a non-adaptive algorithm requiring a minimum of ten spikes with a maximum inter-spike interval of 100 ms. Only the wells that exhibited bursting activity were included in this analysis.

ROS, GSH and Caspase 3/7 Assay.

All luminescence assays were purchased from Promega (ROS-Glo, GSH-Glo and Caspase3/7-Glo) and performed according to manufacturer's instructions. Briefly, 40,000 cells were plated in each well, in sixtuplicate, in a white 96 well plate (Corning) and maintained until 80% confluence is reached. An extra set of 4 wells is used for normalization of cell viability by Alamar Blue (Life Technologies) performed as recommended by manufacturer's instructions. Once cells were ready, media was changed and kept for 48 hours until experiments were performed. The alamar blue assay was performed at the same end point as the luminescence assay and its results were used to normalize the relative luminescence intensity signal from the plate reader (Perkin Elmer plate reader).

Chromatin Immunoprecipitation (ChIP) Assay.

Chromatin immunoprecipitation (ChIP) assay was performed using the ChIP-IT Express Enzymatic kit (Active Motif, Carlsbad, Calif., USA). Details on this assay can be found elsewhere (Nageshappa, et al., 2015).

RTT Neuronal Cultures have Less Synaptogenesis and Altered Network Activity.

In order to generate human RTT-iPSC models without interference from X-chromosome inactivation (Marchetto et al., 2010) and to ensure a homogenous population of MeCP2 mutant cells, skin fibroblasts from two rare male patients, each carrying a novel MECP2 mutation (Q83X and N126I), using their respective unaffected fathers as experimental controls, were reprogramed (FIG. 1A; FIGS. 5A-D and Table 1). In addition, a MECP2-mutant isogenic cell line was generated using CRISPR/Cas9 genome editing (RTT/C9 and Ctrl/C9) (FIG. 1A; FIGS. 5E-L and Tables 2 and 3). Also, a patient cell line with an early stop codon mutation (Q83X), restoring MeCP2 protein levels to normal (rQ83X referred as rRTT) was obtained (FIG. 5M). The pan-neuronal differentiation protocol based on the withdrawal of growth factors from the neural progenitor cells (NPCs) generates mostly forebrain glutamatergic cortical neurons in a mixed population consisting of approximately 50-60% CD24 positive (neurons) and the other 40-50% of cells are GFAP positive (glial lineage) (FIGS. 5N-5R). No difference was observed in the ratio of neurons to glial cells amongst all lines used for this study. Phenotypically, the neurons derived from the male RTT patient cohort reproduced the findings previously described with RTT female lines (Marchetto et al., 2010), including reduced nuclei volume and area (FIG. 1B), defects in the gene expression of neurotrophic factors and insulin growth factor 1 (IGF-1) signaling, glutamate transport and synaptic proteins in RTT-derived cells (FIG. 1C). Moreover, a significant decrease in synaptogenesis, measured by immunofluorescence co-localization of pre-synaptical vesicle SYN1 and Homerl (post-synaptical vesicle) puncta was identified in neuronal processes (FIGS. 1D and 1E; FIG. 5S-5T). Some of these synaptic defects were rescued using IGF-1, as previously demonstrated in Marchetto et al., 2010, which increased the levels of SYN1 and PSD95 (FIG. 5U).

To physiologically test neuronal spontaneous activity from a network perspective, NPCs were seeded and induced to undergo neuronal differentiation in multi-electrode array (MEA) plates (FIG. 1F). Neuronal development can be accompanied by changes in electrical fields associated with the increase of synaptic activity. RTT-derived neuronal networks have a reduced number of spikes per second (spike rate) compared to control cells, and this spike rate was rescued when the MECP2 mutation was genetically reverted (FIGS. 1G-1I). Furthermore, control-derived cells were able to establish a complex network more efficiently when compared to RTT-derived cells, measured by network bursts, where multiple electrode channels show burst activity simultaneously (FIG. 1I). RTT-derived neuronal cell populations presented less synchronized bursts when compared to control cells. It is hypothesized that the decrease in RTT neuronal cell synchronization could be suggestive of the presence of possible neurotoxins present in the RTT environment, accumulated from mutated astrocytes.

Cellular and Molecular Defects in RTT Astrocytes Affects Neuronal Homeostasis.

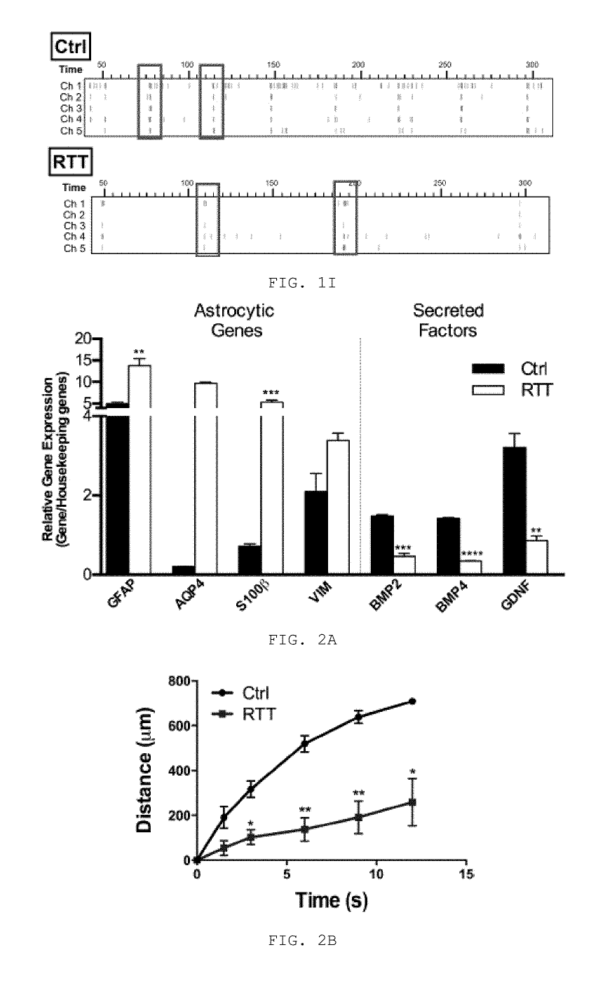

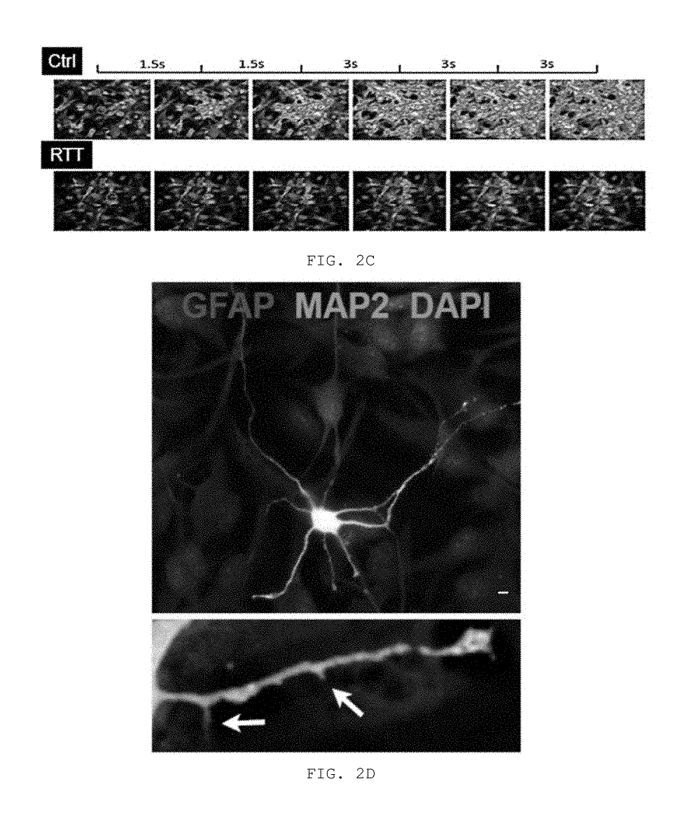

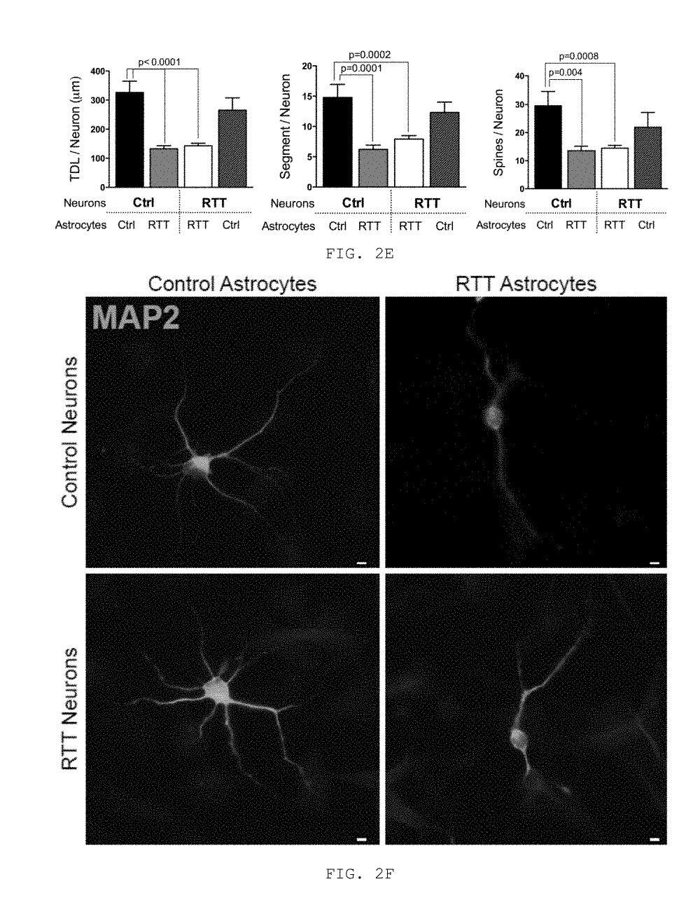

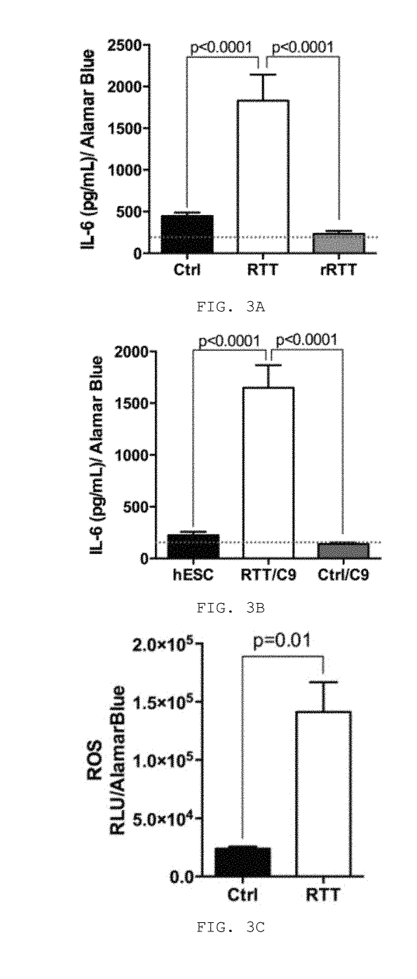

Using the astrocyte differentiation protocol, astrocytes from both RTT and control iPSCs were derived and analyzed for gene expression (FIG. 2A; FIGS. 6A-F). Compared with control cells, RTT astrocytes expressed lower levels of secreted factors such as bone morphogenetic proteins (BMPs) and glial cell line-derived neurotrophic factor (GDNF), which are crucial for gliogenesis and neuronal survival, respectively. To evaluate a well-established physiological astrocyte response, calcium signaling was measured upon mechanical stimulation. In contrast to controls, RTT astrocytes failed to propagate calcium waves over time following mechanical stimulation (FIGS. 2B and 2C).

To determine the role of astrocytes in RTT neuronal morphology growth, co-cultured independently generated human neurons and astrocytes were obtained. Using magnetic sorting to deplete non-neuronal cells (CD184 and CD44 positive), enriched cortical glutamatergic iPSC-derived neurons were seeded on a monolayer of astrocytes that were maintained in co-culture for two weeks (FIG. 6G). The morphology of spines was then quantified, which are important indicators of effective neuronal development and are often compromised in neurodevelopmental disorders, including RTT (Armstrong et al., 1998; Fiala et al., 2002) (FIG. 2D; FIG. 6H). In the presence of control-derived astrocytes, control neurons exhibited longer and more branched dendrites, with twice as many dendritic spines as found in RTT neurons cultured with RTT-derived astrocytes (FIGS. 2E and 2F; FIG. 6I). In contrast, RTT-derived astrocytes negatively affected the dendritic morphology of control neurons, significantly decreasing dendritic length, the total number of dendritic spines and branching complexity of dendritic arbor by 2.4-fold compared to control neurons. In addition, RTT-derived astrocytes reduced the total number of dendritic spines (FIG. 2E). RTT neurons plated with RTT astrocytes lacked the complex dendritic arborization observed in control neurons plated with control-derived astrocytes, as demonstrated by the near absence of higher order dendritic segments (fifth and sixth order of segments from the cell body), which suggests premature dendritic growth arrest (Armstrong et al., 1995) (FIG. 6J). Moreover, the spine distribution across dendritic segments indicated an incomplete maturation of the synaptic inputs onto the dendrites of RTT neurons, with approximately 80% of the spines occurring on the ontologically earliest segments (i.e., first- and second-order segments) (FIG. 6K). Control neurons grown in the presence of RTT-derived astrocytes also displayed compromised RTT-like neuronal morphology, such as a reduced dendrite number, less extensive dendritic arborization and reduced spine number per order of segments, with 48% of the spines being distributed in first-order segments. Similar observations were previously reported in post-mortem analysis of human brain from patients with RTT and other developmental disorders (Armstrong et al., 1998; Fiala et al., 2002). Attempts were then made to rescue the neuronal deficits by co-culturing RTT neurons with control-derived astrocytes. Remarkably, RTT neurons cultured under these conditions showed a significant increase in both dendrite and spine number, with a total dendritic length and dendritic arborization that was similar to that of control neurons (FIGS. 2E and 2F; FIGS. 6I-K).

Increased Expression and Secretion of IL-6 by RTT Astrocytes.

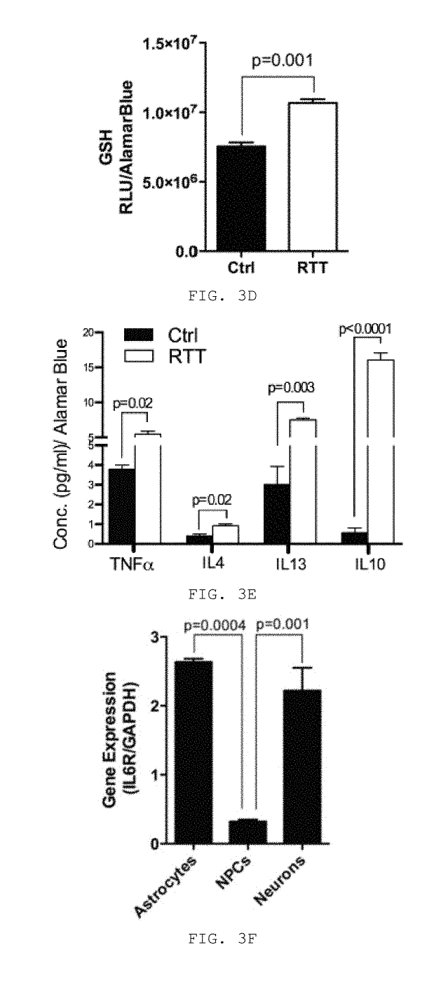

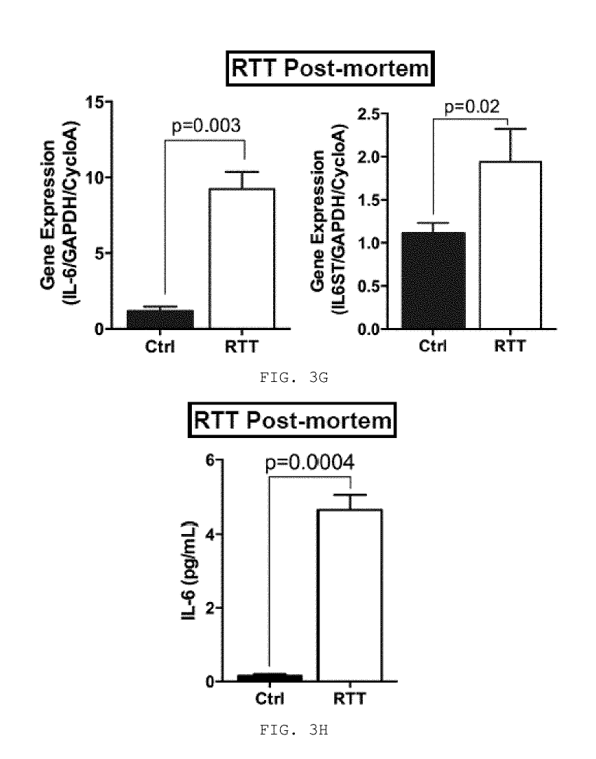

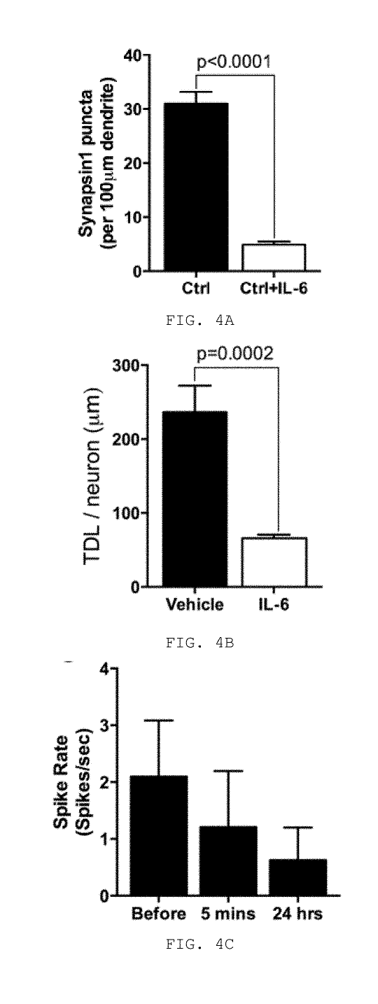

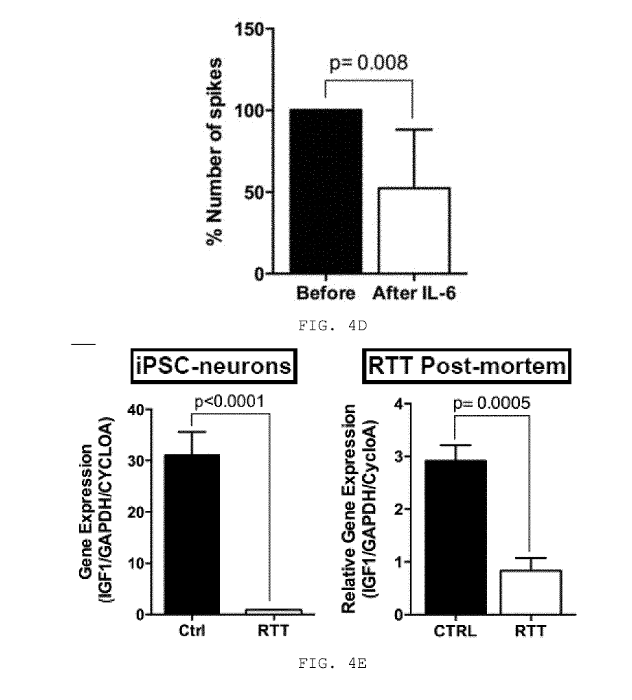

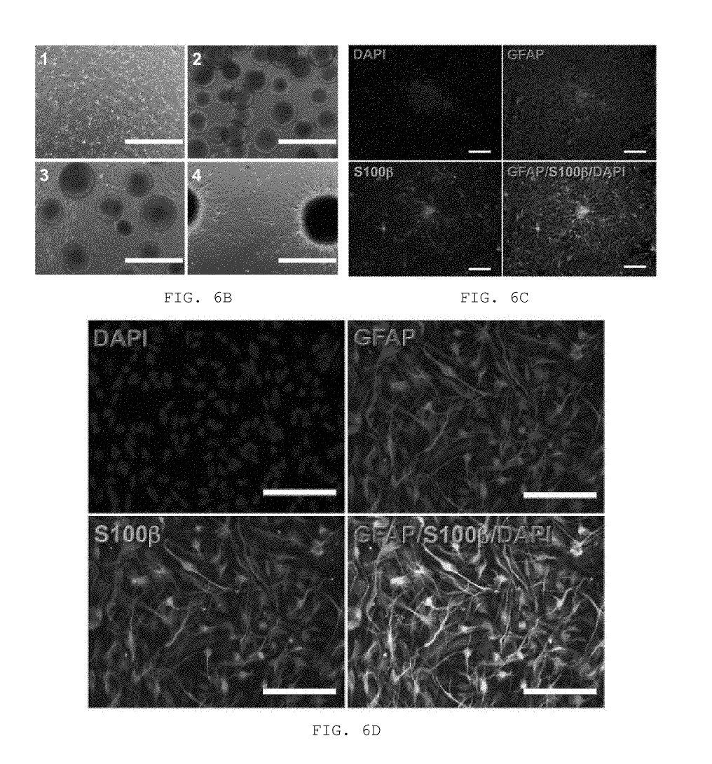

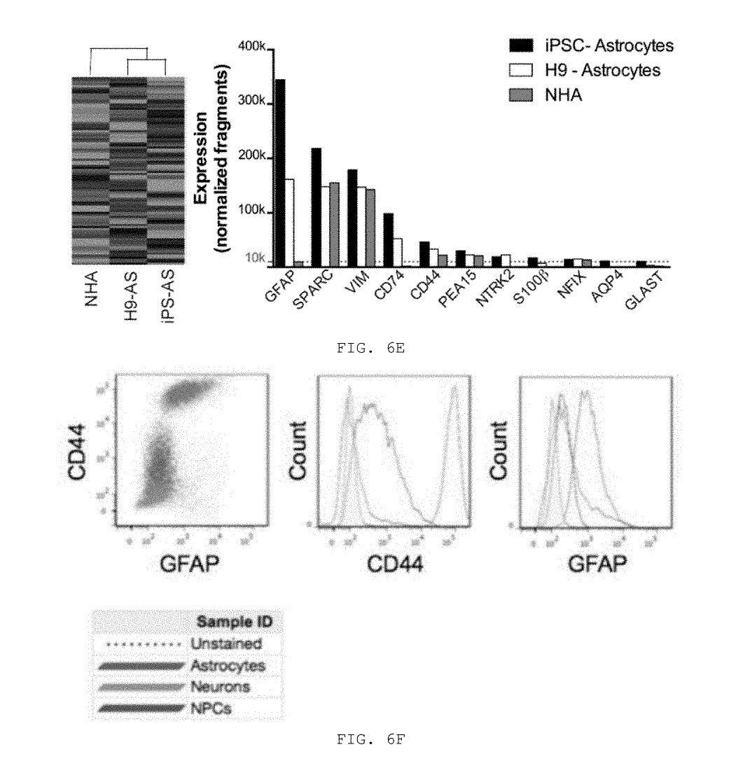

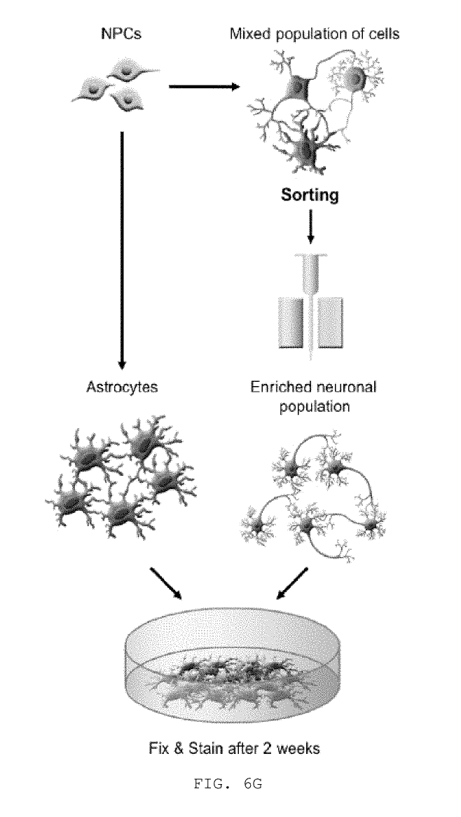

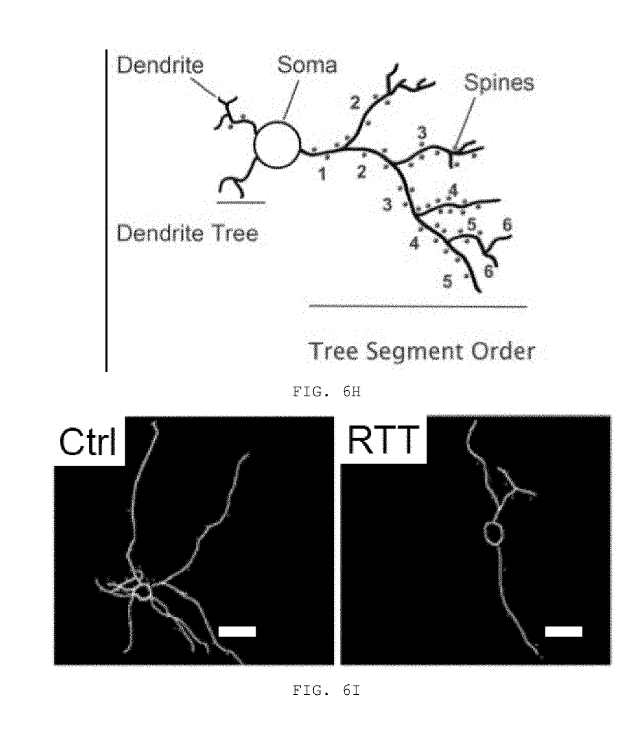

Cytokines are important signaling molecules responsible for cell maturation and inflammation during neurodevelopment (Deverman and Patterson, 2009). IL-6 is a major a pro-inflammatory cytokine, widely implicated with neurodevelopment, neuronal maturation, and autism spectrum disorders (ASD) (Campbell, 1998; Deverman and Patterson, 2009; Vallieres et al., 2002; Van Wagoner et al., 1999; Wei et al., 2013). Thus, experiments were performed to investigate if IL-6 secretion is impaired in RTT-derived astrocytes. Using chromatin immunoprecipitation (ChIP), MeCP2 was observed to occupy the promoter region of IL-6, indicating a possible role of MeCP2 in regulating IL-6 transcription (FIG. 7A). Next, the levels IL-6 released in the media was measured, from human astrocytes, in the absence of exogenous cytokine stimulation and found a significant increase in IL-6 levels from RTT cells compared to controls (FIGS. 3A and 3B). Increased IL-6 levels are associated with cellular oxidative stress (Erta et al., 2012). In fact, significant elevated reactive oxygen species (ROS) levels were observed in RTT astrocytes (FIG. 3C), an event usually followed by the increase in astrocytic production of the anti-oxidant molecule glutathione (Dringen and Hirrlinger, 2003) (FIG. 3D). Discrepancies were also observed in the release of TNFα, IL-4, IL-10 and IL-13 in RTT astrocytes compared to controls, although none of the levels identified were as elevated and significant as the IL-6 level in RTT astrocyte cultures (FIG. 3E). These interleukins are usually released upon bacteria lipopolysaccharides (LPS) or TNFα stimulation in human hematopoietic-derived cells, acting as anti-inflammatory cytokines (IL-4, IL-10 and IL-13) (Marie et al., 1996), although this pathway remains poorly understood in human astrocytes. Moreover, human iPSC-derived neural cells express several cytokine receptors, including the IL-6 receptor alpha (IL6R) that is expressed in both neurons and astrocytes (FIG. 3F; FIG. 7B). No significant differences were found in the amount of secreted IL-6 levels between RTT and RTT/C9 astrocytes, NPCs or neurons, and neither in Caspase 3/7 levels between control and RTT astrocytes (FIGS. 7C and D). The presence of IL-6 in RTT astrocyte cultures was also validated in the only post-mortem brain tissue available from a young male RTT patient. A five-fold increase in IL-6 gene expression, and a two-fold increase in the expression of the gp130 receptor (IL6ST) was identified when compared with an age-matched control sample (FIG. 3G). More importantly, ELISA of the total protein lysate from the cortex showed a five-fold increase in the RTT IL-6 protein level compared to control (FIG. 3H).

Elevated IL-6 Levels Impair Neuronal Physiology.

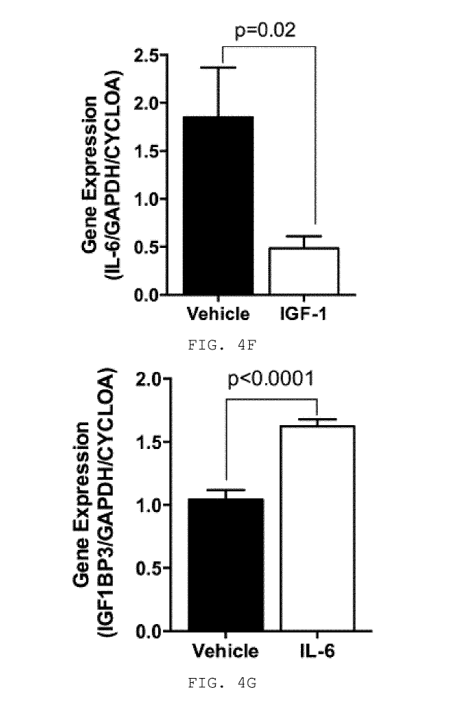

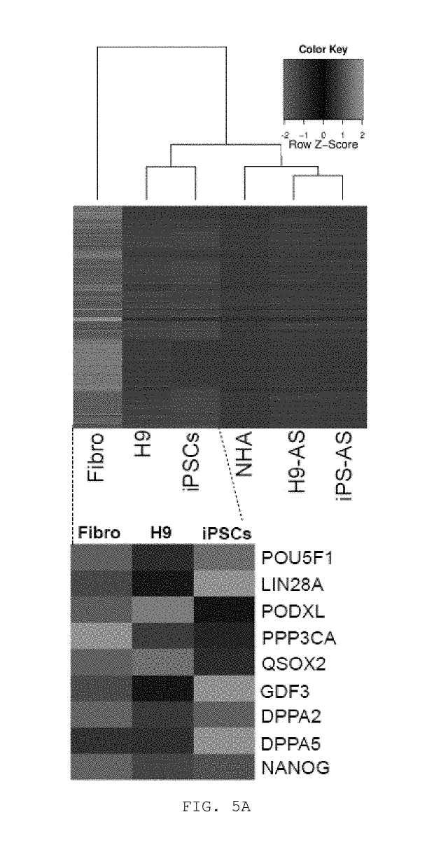

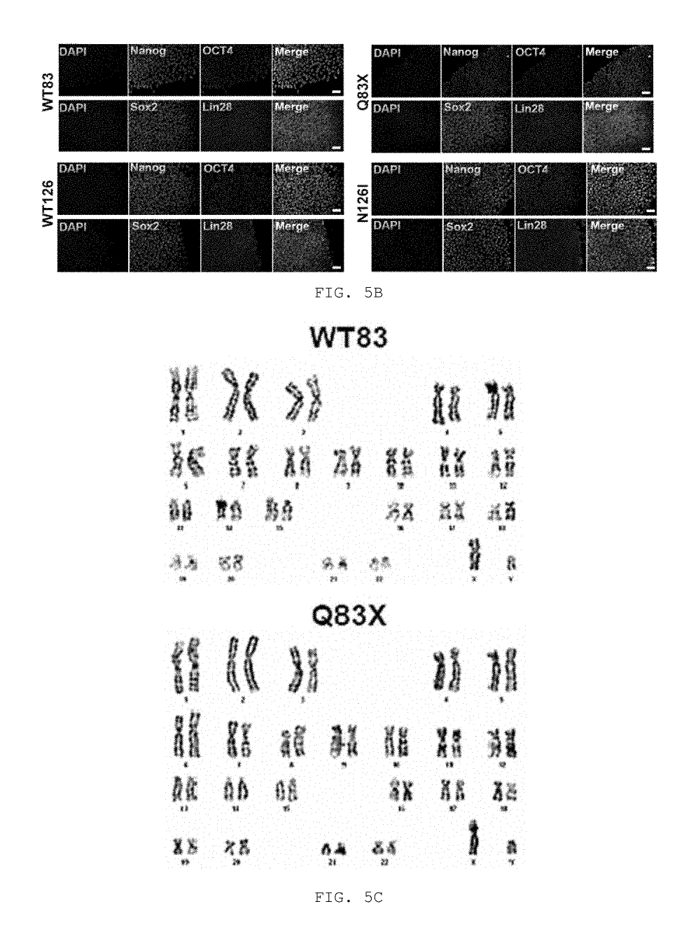

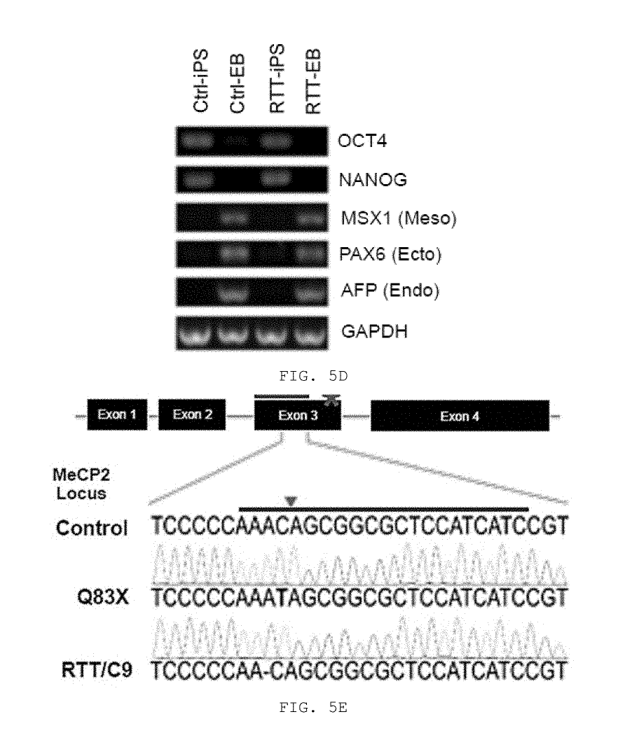

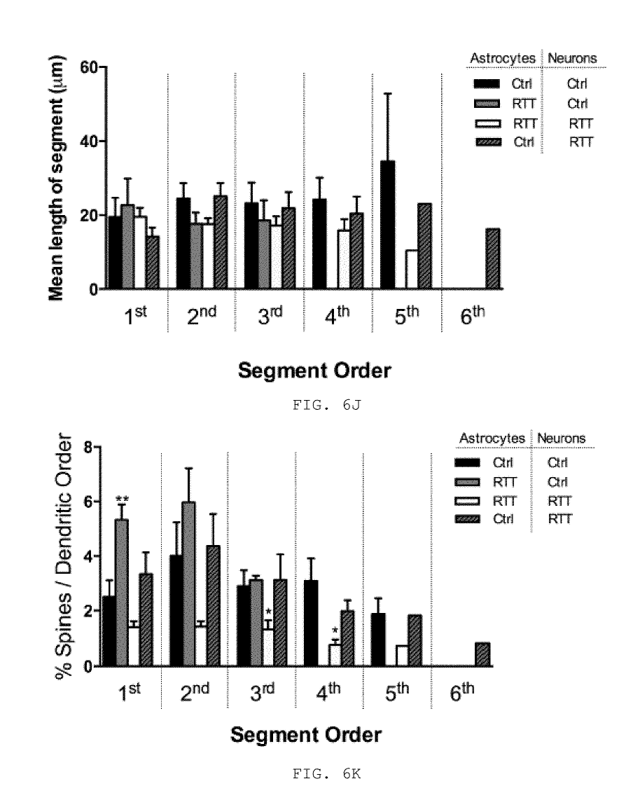

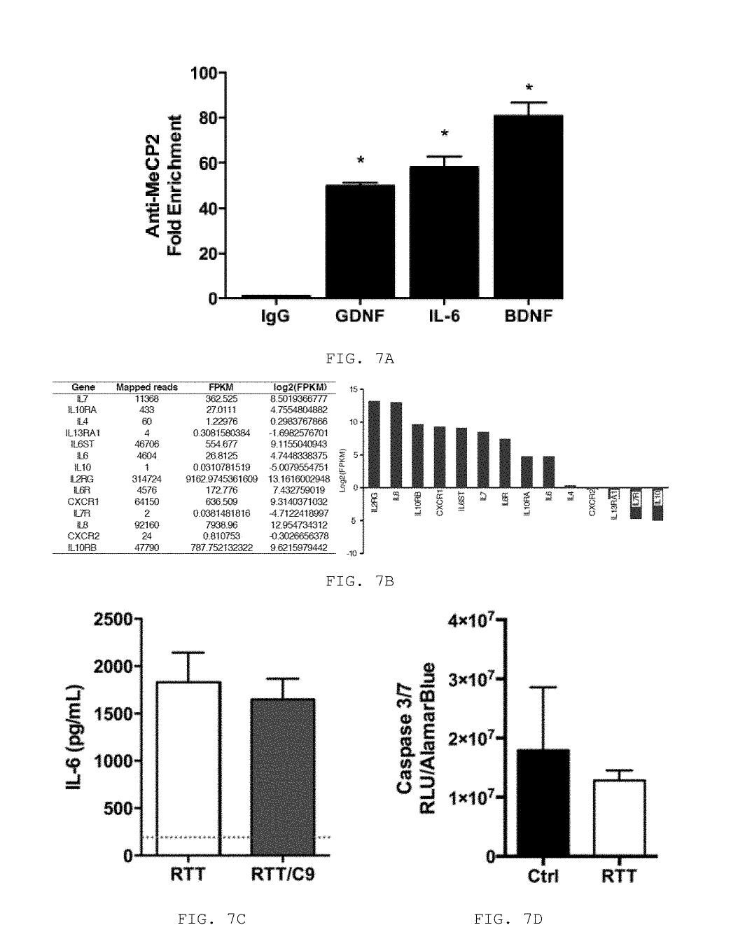

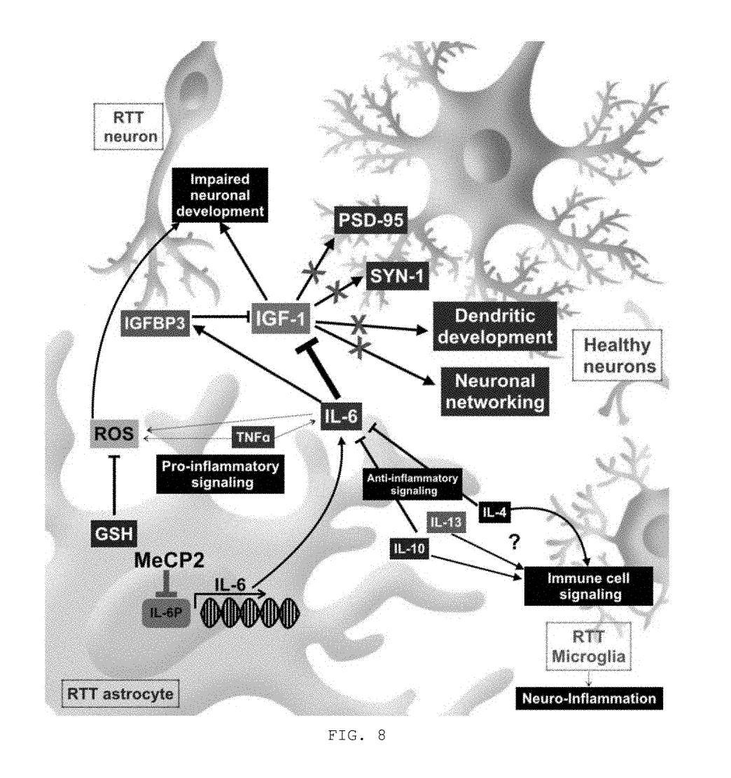

Next, experiments were performed to determine whether elevated exogenous levels of IL-6 release from RTT astrocytes can be toxic to human iPSC-derived neuronal networks. When recombinant human IL-6 (rhIL-6) was added to the culture media of control neurons at the same concentration found in RTT astrocytes (2 ng/mL) over the course of two weeks, a significant decrease in levels of SYN1 puncta (FIG. 4A), and a significant reduction of the total dendritic length of neurons in comparison to vehicle treated cultures (FIG. 4B) was observed. Healthy control neurons that were differentiated in MEA plates decrease their spontaneous activity when rhIL-6 was added to their media (FIGS. 4C and 4D). It was hypothesized that these maturation deficits could arise from the fact that IL-6 can down-regulate IGF-1 (De Benedetti et al., 2001; Nemet et al., 2006). Both RTT-derived neurons and the post-mortem sample showed down-regulation of IGF-1 expression (FIG. 4E). Treatment of IGF-1 for two weeks reduced IL-6 expression in control cells compared to vehicle (FIG. 4F). The decrease of IGF-1 available to neurons is likely related to an increase in the levels of IGFBP3, the main carrier of IGF-1 (De Benedetti et al., 2001; Nemet et al., 2006). Consistently, the addition of IL-6 resulted in an increase of expression of IGFBP3 in both iPSC-derived control and RTT neurons (FIG. 4G and FIG. 8).

Accordingly, the disclosure has provided a series of exemplary embodiments. Alterations, modification, and improvements are intended to be part of this disclosure, and are intended to be within the spirit and scope of the disclosure. Accordingly, the foregoing description and drawings are by way of example only.

1. A cell culture method of generating astrocytes comprising:
culturing neuronal precursor cells (NPCs) to confluence in media contain neuronal growth factor (NGF);
washing and incubating the NPCs with dPBS;
scraping the NPCs to form neurospheres in media containing NGF;
dissociating, plating and culturing the neurospheres under shaking conditions until neurospheres are well formed;
adding ROCK inhibitor in FGF-free NGF containing media and culturing for 1 to 3 days;
passaging and culturing the neurospheres in FGF-free NGF media without ROCK inhibitor for about 1 week;
subculturing the neurospheres in astrocyte media for about 2 weeks with shaking; and
isolating astrocytes that project outside of the spheres to populate the culture plate.

2. The method of claim 1, wherein the NPCs are obtained from induced pluripotent stem cells.

3. The method of claim 1, wherein the induced pluripotent stem cells are obtained from fibroblasts.

4. The method of claim 3, wherein the fibroblasts are obtained from a subject having a neuronal disease, disorder or syndrome.

5. The method of claim 4, wherein the neuronal disease, disorder or syndrome is selected from the group consisting of autism, RETT syndrome, schizophrenia, Fragile X syndrome, Angelman syndrome and Timothy syndrome.

6. The method of claim 2, wherein in the induced pluripotent stem cells are cultured to obtain embryoid bodies and wherein the embryoid bodies are cultured with dual SMAD inhibitors to obtain NPCs.

7. The method of claim 5, wherein the dual SMAD inhibitors comprise Noggin and SB431542.

8. The method of claim 1, wherein the media containing NGF comprises DMEM/F12 media supplemented with 0.5×N2, 0.5× Gem21 supplement, 20 ng/mL of FGF and 1% penicillin/streptomycin.

9. A population of astrocytes obtained by the method of claim 1.

10. A method of screening for an agent that modulates astrocyte activity comprising contacting the population of astrocytes of claim 9, with the agent and measuring a change in the astrocytes' spike activity and/or synaptic activity.

11. The method of claim 10, wherein the population of astrocytes are obtained from a subject having a neuronal disease, disorder or syndrome is selected from the group consisting of autism, RETT syndrome, schizophrenia, Fragile X syndrome, Angelman syndrome and Timothy syndrome.

12. A method comprising:
contacting a human somatic cell obtained from a subject with at least one retroviral vector, wherein each of the at least one retroviral vectors comprises one or more polynucleotides encoding at least four de-differentiation factors selected from the group consisting of a (i) KLF4, (ii) OCT4, (iii) SOX2, (iv) c-MYC or n-MYC, and (v) NANOG,
culturing the somatic cell to express the de-differentiation factor;
selecting cells displaying a pluripotent phenotype;
subculturing the selected cells to obtain an enriched population of de-differentiated induced pluripotent stem cells (iPSCs);
culturing the iPSCs to form embryoid bodies (EBs);
culturing the EBs in N2 media with dual SMAD inhibitors with constant shaking and selecting rosettes for subculture;
subculturing the rosettes in NGF media until NPCs are formed;
changing the NGF media as needed until neurospheres are formed to obtain a neurosphere culture;
adding Rock inhibitor to the neurospheres culture for 1-3 days concomitant with the removal of FGF from the media;
removing Rock inhibitor and culturing the neurospheres in neuronal media without FGF for about one week;
washing the neurospheres and culturing the neurospheres in astrocyte growth media for about two weeks with shaking;
plating neurospheres with astrocyte projections; and
isolating astrocytes.

13. The method of claim 12, further comprising subculturing the isolated astrocytes.

14. A method of screening for an agent that modulates astrocyte activity comprising contacting the isolated astrocytes of claim 12, with the agent and measuring a change in the astrocytes' spike activity and/or synaptic activity.

15. The method of claim 12, wherein the isolated astrocytes are obtained from a subject having a neuronal disease, disorder or syndrome is selected from the group consisting of autism, RETT syndrome, schizophrenia, Fragile X syndrome, Angelman syndrome and Timothy syndrome.


SEQUENCE LISTING (3 sequences)

SEQ ID NO: 1 - DNA, 30 bp, Artificial Sequence
  MECP2 Control Locus
tcccccaaacagcggcgctccatcatccgt

SEQ ID NO: 2 - DNA, 30 bp, Artificial Sequence
  MECP2 mutation Q83X
tcccccaaatagcggcgctccatcatccgt

SEQ ID NO: 3 - DNA, 29 bp, Artificial Sequence
  MECP2 mutation RTT/C9
tcccccaacagcggcgctccatcatccgt